SEQUENCE LISTING (54 sequences)

SEQ ID NO: 1 - protein, 15 aa, Artificial Sequence
  Description of Artificial Sequence Synthetic peptide
DIQMTQSPSSLSASV

SEQ ID NO: 2 - protein, 14 aa, Artificial Sequence
  Description of Artificial Sequence Synthetic peptide
GDRVTITCRASQDV

SEQ ID NO: 3 - protein, 11 aa, Artificial Sequence
  Description of Artificial Sequence Synthetic peptide
NTAVAWYQQKP

SEQ ID NO: 4 - protein, 11 aa, Artificial Sequence
  Description of Artificial Sequence Synthetic peptide
GKAPKLLIYSA

SEQ ID NO: 5 - protein, 9 aa, Artificial Sequence
  Description of Artificial Sequence Synthetic peptide
SFLYSGVPS

SEQ ID NO: 6 - protein, 8 aa, Artificial Sequence
  Description of Artificial Sequence Synthetic peptide
RFSGSRSG

SEQ ID NO: 7 - protein, 12 aa, Artificial Sequence
  Description of Artificial Sequence Synthetic peptide
TDFTLTISSLQP

SEQ ID NO: 8 - protein, 13 aa, Artificial Sequence
  Description of Artificial Sequence Synthetic peptide
EDFATYYCQQHYT

SEQ ID NO: 9 - protein, 14 aa, Artificial Sequence
  Description of Artificial Sequence Synthetic peptide
TPPTFGQGTKVEIK

SEQ ID NO: 10 - protein, 15 aa, Artificial Sequence
  Description of Artificial Sequence Synthetic peptide
TPPTFGQGTKVEIKR

SEQ ID NO: 11 - protein, 14 aa, Artificial Sequence
  Description of Artificial Sequence Synthetic peptide
EVQLVESGGGLVQP

SEQ ID NO: 12 - protein, 15 aa, Artificial Sequence
  Description of Artificial Sequence Synthetic peptide
GGSLRLSCAASGFNI

SEQ ID NO: 13 - protein, 12 aa, Artificial Sequence
  Description of Artificial Sequence Synthetic peptide
KDTYIHWVRQAP

SEQ ID NO: 14 - protein, 13 aa, Artificial Sequence
  Description of Artificial Sequence Synthetic peptide
GKGLEWVARIYPT

SEQ ID NO: 15 - protein, 12 aa, Artificial Sequence
  Description of Artificial Sequence Synthetic peptide
NGYTRYADSVKG

SEQ ID NO: 16 - protein, 10 aa, Artificial Sequence
  Description of Artificial Sequence Synthetic peptide
RFTISADTSK

SEQ ID NO: 17 - protein, 12 aa, Artificial Sequence
  Description of Artificial Sequence Synthetic peptide
NTAYLQMNSLRA

SEQ ID NO: 18 - protein, 15 aa, Artificial Sequence
  Description of Artificial Sequence Synthetic peptide
EDTAVYYCSRWGGDG

SEQ ID NO: 19 - protein, 17 aa, Artificial Sequence
  Description of Artificial Sequence Synthetic peptide
FYAMDYWGQGTLVTVSS

SEQ ID NO: 20 - protein, 19 aa, Artificial Sequence
  Description of Artificial Sequence Synthetic peptide
FYAMDYWGQGTLVTVSSQP

SEQ ID NO: 21 - protein, 5 aa, Artificial Sequence
  Description of Artificial Sequence Synthetic peptide
GGGGS

SEQ ID NO: 22 - protein, 10 aa, Artificial Sequence
  Description of Artificial Sequence Synthetic peptide
GGGGSGGGGS

SEQ ID NO: 23 - protein, 15 aa, Artificial Sequence
  Description of Artificial Sequence Synthetic peptide
GGGGSGGGGSGGGGS

SEQ ID NO: 24 - protein, 20 aa, Artificial Sequence
  Description of Artificial Sequence Synthetic peptide
GGGGSGGGGSGGGGSGGGGS

SEQ ID NO: 25 - protein, 25 aa, Artificial Sequence
  Description of Artificial Sequence Synthetic peptide
GGGGSGGGGSGGGGSGGGGSGGGGS

SEQ ID NO: 26 - protein, 6 aa, Artificial Sequence
  Description of Artificial Sequence Synthetic peptide
TVAAPS

SEQ ID NO: 27 - protein, 7 aa, Artificial Sequence
  Description of Artificial Sequence Synthetic peptide
ASTSGPS

SEQ ID NO: 28 - protein, 6 aa, Artificial Sequence
  Description of Artificial Sequence Synthetic peptide
QAPGKG

SEQ ID NO: 29 - protein, 6 aa, Artificial Sequence
  Description of Artificial Sequence Synthetic peptide
QPKGKA

SEQ ID NO: 30 - protein, 7 aa, Artificial Sequence
  Description of Artificial Sequence Synthetic peptide
VEIKRTV

SEQ ID NO: 31 - protein, 6 aa, Artificial Sequence
  Description of Artificial Sequence Synthetic peptide
ASVGDR

SEQ ID NO: 32 - protein, 8 aa, Artificial Sequence
  Description of Artificial Sequence Synthetic peptide
TVSSASTK

SEQ ID NO: 33 - protein, 6 aa, Artificial Sequence
  Description of Artificial Sequence Synthetic peptide
VQPGGS

SEQ ID NO: 34 - protein, 108 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 35 - protein, 120 aa, Artificial Sequence [sequence omitted]

SEQ ID NO: 36 - protein, 215 aa, Artificial Sequence
  Description of Artificial Sequence Synthetic polypeptide
EVQLVESGGGLVQPGGSLRLSCAASGFNIKDTYIHWVRQAPGKAPKLLIYSASFLYSGVPSRFSGSRSGTDFTLTISSLQPEDFATYYCQQHYTTPPTFGQGTKVEIKRSVGDRVTITCRASQDVNTAVAWYQQKPGKGLEWVARIYPTNGYTRYADSVKGRFTISADTSKNTAYLQMNSLRAEDTAVYYCSRWGGDGFYAMDYWGQGTLVTVSS

SEQ ID NO: 37 - protein, 216 aa, Artificial Sequence
  Description of Artificial Sequence Synthetic polypeptide
DIQMTQSPSSLSASVGDRVTITCRASQDVNTAVAWYQQKPGKGLEWVARIYPTNGYTRYADSVKGRFTISADTSKNTAYLQMNSLRAEDTAVYYCSRWGGDGFYAMDYWGQGTLVTVSSQPGGSLRLSCAASGFNIKDTYIHWVRQAPGKAPKLLIYSASFLYSGVPSRFSGSRSGTDFTLTISSLQPEDFATYYCQQHYTTPPTFGQGTKVEIKR

SEQ ID NO: 38 - protein, 203 aa, Artificial Sequence
  Description of Artificial Sequence Synthetic polypeptide
GKAPKLLIYSASFLYSGVPSRFSGSRSGTDFTLTISSLQPEDFATYYCQQHYTTPPTFGQGTKVEIKRSVGDRVTITCRASQDVNTAVAWYQQKPGKGLEWVARIYPTNGYTRYADSVKGRFTISADTSKNTAYLQMNSLRAEDTAVYYCSRWGGDGFYAMDYWGQGTLVTVSSQPGGSLRLSCAASGFNIKDTYIHWVRQAP

SEQ ID NO: 39 - protein, 203 aa, Artificial Sequence
  Description of Artificial Sequence Synthetic polypeptide
GKGLEWVARIYPTNGYTRYADSVKGRFTISADTSKNTAYLQMNSLRAEDTAVYYCSRWGGDGFYAMDYWGQGTLVTVSSQPGGSLRLSCAASGFNIKDTYIHWVRQAPGKAPKLLIYSASFLYSGVPSRFSGSRSGTDFTLTISSLQPEDFATYYCQQHYTTPPTFGQGTKVEIKRSVGDRVTITCRASQDVNTAVAWYQQKP

SEQ ID NO: 40 - DNA, 30 bp, Artificial Sequence
  Description of Artificial Sequence Synthetic primer
ggtgtctccatcttccatcgatttatttac

SEQ ID NO: 41 - DNA, 50 bp, Artificial Sequence
  Description of Artificial Sequence Synthetic primer
tcaccgtctcctcgggatccgagcccaaatcttgtgacaaaactcacaca

SEQ ID NO: 42 - DNA, 21 bp, Artificial Sequence
  Description of Artificial Sequence Synthetic primer
aaggaattgagagccgctagc

SEQ ID NO: 43 - DNA, 23 bp, Artificial Sequence
  Description of Artificial Sequence Synthetic primer
cgaggagacggtgaccagggttc

SEQ ID NO: 44 - DNA, 27 bp, Artificial Sequence
  Description of Artificial Sequence Synthetic primer
tttttgctaatcccttttgtgtgctga

SEQ ID NO: 45 - DNA, 23 bp, Artificial Sequence
  Description of Artificial Sequence Synthetic primer
tgaccacacggtacgtgctgttg

SEQ ID NO: 46 - DNA, 896 bp, Artificial Sequence
  Description of Artificial Sequence Synthetic polynucleotide
gaaaaaaggaattgagagccgctagcgccaccatgaggctccctgctcagctcctggggctgctaatgctctgggtcccaggctccagtggggaggttcagctggtggagtctggcggtggcctggtgcagccagggggctcactccgtttgtcctgtgcagcttctggcttcaacattaaagacacctatatacactgggtgcgtcaggccccgggaaaagctccgaaactactgatttactcggcatccttcctctactctggagtcccttctcgcttctctggttccagatctgggacggatttcactctgaccatcagcagtctgcagccggaagacttcgcaacttattactgtcagcaacattatactactcctcccacgttcggacagggtaccaaggtggagatcaaacgttctgtgggcgatagggtcaccatcacctgccgtgccagtcaggatgtgaatactgctgtagcctggtatcaacagaaaccaggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcgggatccgagcccaaatctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtg

SEQ ID NO: 47 - DNA, 899 bp, Artificial Sequence
  Description of Artificial Sequence Synthetic polynucleotide
gaaaaaaggaattgagagccgctagcgccaccatgaggctccctgctcagctcctggggctgctaatgctctgggtcccaggctccagtggggatatccagatgacccagtccccgagctccctgtccgcctctgtgggcgatagggtcaccatcacctgccgtgccagtcaggatgtgaatactgctgtagcctggtatcaacagaaaccaggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcgcagccagggggctcactccgtttgtcctgtgcagcttctggcttcaacattaaagacacctatatacactgggtgcgtcaggccccgggaaaagctccgaaactactgatttactcggcatccttcctctactctggagtcccttctcgcttctctggttccagatctgggacggatttcactctgaccatcagcagtctgcagccggaagacttcgcaacttattactgtcagcaacattatactactcctcccacgttcggacagggtaccaaggtggagatcaaacgtggatccgagcccaaatctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtg

SEQ ID NO: 48 - DNA, 860 bp, Artificial Sequence
  Description of Artificial Sequence Synthetic polynucleotide
gaaaaaaggaattgagagccgctagcgccaccatgaggctccctgctcagctcctggggctgctaatgctctgggtcccaggctccagtgggggaaaagctccgaaactactgatttactcggcatccttcctctactctggagtcccttctcgcttctctggttccagatctgggacggatttcactctgaccatcagcagtctgcagccggaagacttcgcaacttattactgtcagcaacattatactactcctcccacgttcggacagggtaccaaggtggagatcaaacgttctgtgggcgatagggtcaccatcacctgccgtgccagtcaggatgtgaatactgctgtagcctggtatcaacagaaaccaggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcgcagccagggggctcactccgtttgtcctgtgcagcttctggcttcaacattaaagacacctatatacactgggtgcgtcaggccccgggatccgagcccaaatctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtg

SEQ ID NO: 49 - DNA, 860 bp, Artificial Sequence
  Description of Artificial Sequence Synthetic polynucleotide
gaaaaaaggaattgagagccgctagcgccaccatgaggctccctgctcagctcctggggctgctaatgctctgggtcccaggctccagtgggggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcgcagccagggggctcactccgtttgtcctgtgcagcttctggcttcaacattaaagacacctatatacactgggtgcgtcaggccccgggaaaagctccgaaactactgatttactcggcatccttcctctactctggagtcccttctcgcttctctggttccagatctgggacggatttcactctgaccatcagcagtctgcagccggaagacttcgcaacttattactgtcagcaacattatactactcctcccacgttcggacagggtaccaaggtggagatcaaacgttctgtgggcgatagggtcaccatcacctgccgtgccagtcaggatgtgaatactgctgtagcctggtatcaacagaaaccaggatccgagcccaaatctagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtg

SEQ ID NO: 50 - protein, 467 aa, Artificial Sequence
  Description of Artificial Sequence Synthetic polypeptide
MRLPAQLLGLLMLWVPGSSGEVQLVESGGGLVQPGGSLRLSCAASGFNIKDTYIHWVRQAPGKAPKLLIYSASFLYSGVPSRFSGSRSGTDFTLTISSLQPEDFATYYCQQHYTTPPTFGQGTKVEIKRSVGDRVTITCRASQDVNTAVAWYQQKPGKGLEWVARIYPTNGYTRYADSVKGRFTISADTSKNTAYLQMNSLRAEDTAVYYCSRWGGDGFYAMDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 468 aa, Artificial Sequence
  Description of Artificial Sequence Synthetic polypeptide
MRLPAQLLGLLMLWVPGSSGDIQMTQSPSSLSASVGDRVTITCRASQDVNTAVAWYQQKPGKGLEWVARIYPTNGYTRYADSVKGRFTISADTSKNTAYLQMNSLRAEDTAVYYCSRWGGDGFYAMDYWGQGTLVTVSSQPGGSLRLSCAASGFNIKDTYIHWVRQAPGKAPKLLIYSASFLYSGVPSRFSGSRSGTDFTLTISSLQPEDFATYYCQQHYTTPPTFGQGTKVEIKREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 455 aa, Artificial Sequence
  Description of Artificial Sequence Synthetic polypeptide
MRLPAQLLGLLMLWVPGSSGGKAPKLLIYSASFLYSGVPSRFSGSRSGTDFTLTISSLQPEDFATYYCQQHYTTPPTFGQGTKVEIKRSVGDRVTITCRASQDVNTAVAWYQQKPGKGLEWVARIYPTNGYTRYADSVKGRFTISADTSKNTAYLQMNSLRAEDTAVYYCSRWGGDGFYAMDYWGQGTLVTVSSQPGGSLRLSCAASGFNIKDTYIHWVRQAPEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 455 aa, Artificial Sequence
  Description of Artificial Sequence Synthetic polypeptide
MRLPAQLLGLLMLWVPGSSGGKGLEWVARIYPTNGYTRYADSVKGRFTISADTSKNTAYLQMNSLRAEDTAVYYCSRWGGDGFYAMDYWGQGTLVTVSSQPGGSLRLSCAASGFNIKDTYIHWVRQAPGKAPKLLIYSASFLYSGVPSRFSGSRSGTDFTLTISSLQPEDFATYYCQQHYTTPPTFGQGTKVEIKRSVGDRVTITCRASQDVNTAVAWYQQKPEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 122 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSQP

(19) United States
(12) Patent Application Publication
(10) Pub. No.: US 2016/0176983 A1
(43) Pub. Date: Jun. 23, 2016

(54) INTERCALATED SINGLE-CHAIN VARIABLE FRAGMENTS

(71) Applicant: Intrexon Corporation, Blacksburg, VA (US)

(72) Inventors: Lei JIA, Newbury Park, CA (US); Charles REED, Souderton, PA (US); Vinodhbabu KURELLA, Rockville, MD (US)

(21) Appl. No.: 14/971,502

(22) Filed: Dec. 16, 2015

Related U.S. Application Data
(60) Provisional application No. 62/132,960, filed on Mar. 13, 2015.
(60) Provisional application No. 62/093,090, filed on Dec. 17, 2014.

(51) Int. Cl. C07K 16/32; G01N 33/543

(52) U.S. Cl. CPC C07K 16/32; G01N 33/54393; C07K 2317/622; C07K 2317/567; C07K 2317/14

(57) ABSTRACT

Single chain antibody polypeptides with engineered peptide bond crossovers in the light chain and/or heavy chain variable domains, compositions comprising the same, and methods of making and using the same are provided. The antibody polypeptides can be intercalated (crossover) single chain variable fragments (scFvs) or any antibody frameworks which comprise such scFvs, such as diabodies, bispecific antibodies or bssFvs. The single chain antibody polypeptides may or may not contain a linker. The single chain antibody polypeptides are useful in applications where standard (conventional) scFvs are useful, such as in the development of scFv libraries for screening, as therapeutic antibodies, or in in vitro and in vivo targeting applications.

### REFERENCE TO RELATED APPLICATIONS

This application claims the benefit of U.S. Provisional Application No. 62/132,960, filed Mar. 13, 2015, and U.S. Provisional Application No. 62/093,090, filed Dec. 17, 2014, both of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Dec. 11, 2015, is named 205350_0003_00_US_ST25.txt and is 42,770 bytes in size.

### TECHNICAL FIELD

The field of art to which the disclosure pertains is antibodies; more specifically involving immunoglobulins or antibodies produced via recombinant or synthetic DNA technology.

### BACKGROUND

With more than 30 molecules approved for clinical use, monoclonal antibodies (mAbs) have come of age as therapeutics, and are now the largest class of biological therapies under development. Monoclonal antibodies are large (150 kDa) multimeric proteins containing numerous disulphide bonds and post-translational modifications such as glycosylation. See e.g., Antibodies: A Laboratory Manual (2^{nd} Edition), E. A. Greenfield (Editor), Cold Spring Harbor Laboratory Press (2013). Thus, mAbs are functionally limited by their size (less than optimal pharmacokinetics, tissue accessibility) and high production costs (necessitate the use of very large cultures of mammalian cells followed by extensive purification steps). Moreover, as light chains can self-aggregate when expressed at high levels recombinantly, and a full monoclonal antibody is not feasible for gene-based therapies, alternative antibody scaffolds have been developed to overcome the particular technical hurdles of multi-chain mAb production.

The single-chain variable fragment (scFv) is one of the most widely used antibody scaffolds, which was developed to circumvent problems associated with the assembly of a functional binder from two polypeptide chains that are expressed separately. An scFv is a fusion protein of the variable regions of the heavy (V_{H}) and light chains (V_{L}) of immunoglobulins. The scFv variable regions are connected with a short linker peptide of ten to about 25 amino acids. The linker is usually rich in glycine for flexibility, as well as serine or threonine for solubility, and can either connect the N-terminus of the heavy chain variable domain with the C-terminus of the light chain variable domain, or vice versa. The scFv retains the specificity of the original immunoglobulin, despite removal of the constant regions and the introduction of the linker. See, e.g., “Antibody Structure” at www.bioatla.com/wp-content/uploads/Appendix_antibodystructure.pdf. This standard scFv configuration has been used for at least 20 years without any significant change.

Yet the scFv has several drawbacks when compared to full length antibodies and other scaffolds such as Fabs (Fragments antibody binding). It is known that the conserved segment in the Fab molecule stabilizes the native structure, which is similar to its structure in the whole antibody. In contrast, the cloned scFv is less stable than the Fab and tends to aggregate rather than form a heterodimer. Variable region sequence, linker length and linker composition can all impact overall efficiency of scFv secretion as well as binding affinities. In particular, the peptide linker can damage an scFv's binding conformation and binding kinetics because it may obscure the antigen binding site. Standard scFv molecules typically have long peptide linkers, but it is known that this configuration is not always ideal for some scFv's and that linker design can at times greatly affect scFv function. Finally, ScFv's typically bind with slightly lower affinities than full-length antibodies. However, the advantage of a single-gene expression platform continues to make the scFv a high-value target in both the therapeutic antibody and the research antibody space.

### SUMMARY

A novel single chain polypeptide antibody framework (platform) is provided. More particularly, genetically engineered, single chain antibodies comprising reorganized peptide bonds (or “crossovers”) in the variable domains resulting in intercalated single chain variable fragment (xscFv) antibodies, compositions comprising the same, and methods of making and using the same are provided.

Single chain antibodies that do not require the use of long linkers or components other than antibody variable domains are provided. Elimination or reduction of linker sequences creates a molecular design that may be applied to screening libraries with diverse immunoglobulin scaffolds. Intercalated single chain antibody polypeptides may comprise a reduced linker, or no linker, which may eliminate interruption or hindrance in scFv assembly/polypeptide folding. Moreover, the lack of long peptide linkers may reduce cross-association of V domains between molecules such that intracellular or extracellular protein aggregation is reduced. Single chain antibody polypeptides may also be completely human in origin, thereby enhancing therapeutic potential.

The antibody polypeptides comprise a light chain variable domain (V_{L}) and a heavy chain variable domain (V_{H}), and can, but need not comprise a linker. Exemplary antibody polypeptides are in the form of crossover scFvs (“xscFvs”), which are intercalated single chain variable fragments with crossovers in their variable domains. The antibody polypeptides can be conjugated to other entities, such as Fc domains or drugs. Alternatively, the antibody polypeptides can include additional variable domains and form diabodies. The antibody polypeptides can be bispecific antibodies or bispecific minibodies that comprise xscFv and one or more additional variable domains.

The antibody polypeptides can serve as supporting frameworks for the presentation of polypeptide libraries. They can be subjected to powerful in vitro or in vivo selection and evolution strategies, enabling the isolation of high-affinity binding reagents. The antibody polypeptides can also be used in any applications where scFvs are otherwise used, such as affinity purification, protein microarray technologies, bioimaging, enzyme inhibition, flow cytometry, in a biosensor to bind a specific molecule or antigen, immunohistochemistry, as antigen-binding domains of artificial T cell receptors, as a conjugate to a drug for targeting, on a chimeric antigen receptor to direct cell killing, and as part of a bispecific engineered antibody such as bispecific single-chain Fvs (bsscFvs) to link target cells and effector cells. Moreover, the antibody polypeptide can be used anywhere where a full-length monoclonal antibody 1) can be used or 2) is impractical, not preferred, or impossible to use.

A single chain antibody polypeptide can comprise a heavy chain variable domain and a light chain variable domain, wherein the antibody polypeptide comprises at least one interdomain crossover, at least one intradomain crossover, or at least one intradomain crossover and at least one interdomain crossover, wherein each crossover is an engineered peptide bond producing an intercalated structure (arrangement or organization) selected from the group consisting of:
a) at least one portion of the heavy chain variable domain intercalated (inserted) into the light chain variable domain;
b) at least one portion of the light chain variable domain intercalated (inserted) into the heavy chain variable domain;
c) at least one portion of the light chain variable domain intercalated (inserted) into a different portion of the light chain variable domain; and
d) at least one portion of the heavy chain variable domain intercalated (inserted) into a different portion of the heavy chain variable domain.

Embodiments also comprise a single chain antibody polypeptide, comprising at least one intradomain crossover and at least one interdomain crossover.

Embodiments also comprise a single chain antibody polypeptide, comprising at least two intradomain crossovers.

Embodiments also comprise a single chain antibody polypeptide, wherein each crossover is introduced between variable domain regions which are within approximately 9-12 Å of each other prior to intercalation (insertion).

Embodiments also comprise a single chain antibody polypeptide, wherein the intercalated (inserted) portion comprises at least one variable domain immunoglobulin beta strand.

Embodiments also comprise a single chain antibody polypeptide, wherein said at least one variable domain immunoglobulin (V_{H} or V_{L}) beta strand is intercalated (inserted) within a different region (portion) of the same variable domain (V_{H} or V_{L}, respectively).

Embodiments also comprise a single chain antibody polypeptide, wherein the polypeptide comprises immunoglobulin beta strands (1)-(17): 1) A_{H}; 2) B_{H}; 3) C_{H}; 4) C′_{L}; 5) C″_{L}; 6) D_{L}; 7) E_{L}; 8) F_{L}; 9) G_{L}; 10) B_{L}; 11) C_{L}; 12) C′_{H}; 13) C″_{H}; 14) D_{H}; 15) E_{H}; 16) F_{H}; 17) G_{H}, and wherein β-strands (1)-(17) are arranged sequentially from the N to the C terminus in the polypeptide.

Embodiments also comprise a single chain antibody polypeptide, wherein the polypeptide comprises immunoglobulin β-strands (1)-(17): 1) A_{L}; 2) B_{L}; 3) C_{L}; 4) C′_{H}; 5) C″_{H}; 6) D_{H}; 7) E_{H}; 8) F_{H}; 9) G_{H}; 10) B_{H}; 11) C_{H}; 12) C′_{L}; 13) C″_{L}; 14) D_{L}; 15) E_{L}; 16) F_{L}; 17) G_{L}, and wherein β-strands (1)-(17) are arranged sequentially from the N to the C terminus in the polypeptide.

Embodiments also comprise a single chain antibody polypeptide, wherein the polypeptide comprises immunoglobulin β-strands (1)-(16): 1) C′_{L}; 2) C″_{L}; 3) D_{L}; 4) E_{L}; 5) F_{L}; 6) G_{L}; 7) B_{L}; 8) C_{L}; 9) C′_{H}; 10) C″_{H}; 11) D_{H}; 12) E_{H}; 13) F_{H}; 14) G_{H}; 15) B_{H}; 16) C_{H}, and wherein β-strands (1)-(16) are arranged sequentially from the N to the C terminus in the polypeptide.

Embodiments also comprise a single chain antibody polypeptide, wherein the polypeptide comprises immunoglobulin β-strands (1)-(16): 1) C′_{H}; 2) C″_{H}; 3) D_{H}; 4) E_{H}; 5) F_{H}; 6) G_{H}; 7) B_{H}; 8) C_{H}; 9) C′_{L}; 10) C″_{L}; 11) D_{L}; 12) E_{L}; 13) F_{L}; 14) G_{L}; 15) B_{L}; 16) C_{L}, and wherein β-strands (1)-(16) are arranged sequentially from the N to the C terminus in the polypeptide.

Embodiments also comprise a single chain antibody polypeptide, wherein immunoglobulin β-strands (1)-(17) or (1)-(16) are selected from the group consisting of:
a) A_{L} comprises amino acid residues DIQMTQSPSSLSASV (SEQ ID NO: 1);
b) B_{L} comprises amino acid residues GDRVTITCRASQDV (SEQ ID NO: 2);
c) C_{L} comprises amino acid residues NTAVAWYQQKP (SEQ ID NO: 3);
d) C′_{L} comprises amino acid residues GKAPKLLIYSA (SEQ ID NO: 4);
e) C″_{L} comprises amino acid residues SFLYSGVPS (SEQ ID NO: 5); D_{L} comprises amino acid residues RFSGSRSG (SEQ ID NO: 6);
g) E_{L} comprises amino acid residues TDFTLTISSLQP (SEQ ID NO: 7);
h) F_{L} comprises amino acid residues EDFATYYCQQHYT (SEQ ID NO: 8);
i) G_{L} comprises amino acid residues TPPTFGQGTKVEIK (SEQ ID NO: 9);
j) G_{L} comprises amino acid residues TPPTFGQGTKVEIKR (SEQ ID NO: 10);
k) A_{H} comprises amino acid residues EVQLVESGGGLVQP (SEQ ID NO: 11);
l) B_{H} comprises amino acid residues GGSLRLSCAASGFNI (SEQ ID NO: 12);
m) C_{H} comprises amino acid residues KDTYIHWVRQAP (SEQ ID NO: 13);
n) C′_{H} comprises amino acid residues GKGLEWVARIYPT (SEQ ID NO: 14);
o) C″_{H} comprises amino acid residues NGYTRYADSVKG (SEQ ID NO: 15);
p) D_{H} comprises amino acid residues RFTISADTSK (SEQ ID NO: 16);
q) E_{H} comprises amino acid residues NTAYLQMNSLRA (SEQ ID NO: 17);
r) F_{H} comprises amino acid residues EDTAVYYCSRWGGDG (SEQ ID NO: 18);
s) G_{H} comprises amino acid residues FYAMDYWGQGTLVTVSS (SEQ ID NO: 19); and,
t) G_{H} comprises amino acid residues FYAMDYWGQGTLVTVSSQP (SEQ ID NO: 20).

Embodiments also comprise an antibody framework comprising the single chain antibody polypeptide, wherein the antibody framework is selected from the group consisting of a Fab fragment comprising crossovers, a F(ab′)₂ fragment comprising crossovers, an Fv fragment comprising crossovers, a diabody comprising crossovers, a minibody comprising crossovers, a bispecific antibody comprising crossovers, a bispecific single-chain Fvs (bsscFvs) comprising crossovers, and a chimeric antigen receptor comprising crossovers. Optionally, the single chain antibody polypeptide of the antibody framework further comprises a linker of 1, 2, 3, 4, 5, 6, 7 or 5 amino acid residues.

Embodiments also comprise a single chain antibody polypeptide, wherein the antibody polypeptide is an xscFv.

Embodiments also comprise a single chain antibody polypeptide, wherein the polypeptide further comprises a linker of 1, 2, 3, 4, 5, 6, 7 or 5 amino acid residues.

Embodiments also comprise a nucleic acid encoding a single chain antibody polypeptide or an antibody framework comprising a single chain antibody polypeptide.

Embodiments also comprise a vector comprising a nucleic acid encoding a single chain antibody polypeptide or an antibody framework comprising a single chain antibody polypeptide.

Embodiments also comprise a host cell comprising a nucleic acid encoding a single chain antibody polypeptide or an antibody framework comprising a single chain antibody polypeptide.

Embodiments also comprise a host cell expressing a single chain antibody polypeptide or an antibody framework comprising a single chain antibody polypeptide.

Embodiments also comprise a method of making a single chain antibody polypeptide or an antibody framework comprising a single chain antibody polypeptide comprising culturing a host cell expressing the single chain antibody polypeptide or an antibody framework comprising a single chain antibody polypeptide.

Embodiments also comprise an in vitro method of targeting an antigen comprising contacting an antigen in vitro with a single chain antibody polypeptide or an antibody framework comprising a single chain antibody polypeptide, wherein said single chain antibody polypeptide binds said antigen.

Embodiments also comprise an in vivo method of targeting an antigen comprising contacting an antigen in vivo with a single chain antibody polypeptide or an antibody framework comprising a single chain antibody polypeptide, wherein said single chain antibody polypeptide binds said antigen.

Embodiments also comprise a method of generating a combinatorial or mutagenized library of xscFvs.

Embodiments also comprise a pharmaceutical composition or medicament comprising a single chain antibody polypeptide, an antibody framework comprising a single chain antibody polypeptide, a nucleic acid encoding a single chain antibody polypeptide or an antibody framework comprising a single chain antibody polypeptide, a vector comprising a nucleic acid encoding a single chain antibody polypeptide or an antibody framework comprising a single chain antibody polypeptide, or a host cell comprising a vector or nucleic acid encoding a single chain antibody polypeptide or an antibody framework comprising a single chain antibody polypeptide.

Embodiments also comprise a library of single chain antibody polypeptides, wherein said single chain antibody polypeptides comprise a heavy chain variable domain and a light chain variable domain, wherein the antibody polypeptides comprise at least one interdomain crossover, at least one intradomain crossover, or at least one intradomain crossover and at least one interdomain crossover, wherein each crossover is selected from the group consisting of:
a) at least one portion of a heavy chain variable domain intercalated (inserted) into a light chain variable domain;
b) at least one portion of a light chain variable domain intercalated (inserted) into a heavy chain variable domain;
c) at least one portion of a light chain variable domain intercalated (inserted) into a different portion of the light chain variable domain; and
d) at least one portion of a heavy chain variable domain intercalated (inserted) into a different portion of the heavy chain variable domain.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1E show a series of model standard scFv and crossover scFv (xscFv) models in ribbon diagram format. The blue and red regions correspond to the V_{H} and V_{L}, respectively, of a standard scFv. FIG. 1A depicts a standard scFv configuration. The scFv linker is depicted in yellow. FIG. 1B depicts xscFv configuration 1 without a linker. FIG. 1C depicts in xscFv configuration 2 without a linker. FIG. 1D depicts xscFv configuration 3 without a linker. FIG. 1E depicts xscFv configuration 4 without a linker.

FIG. 2 shows a series of standard scFv and crossover scFv models in a simplified topology diagram format. Light chain variable region beta sheets 1-9 are labeled A-G (red), respectively. Heavy chain variable regions beta sheets 1-9 are labeled A-G (blue), respectively. scFv linkers are depicted in yellow. The standard scFv is labeled scFv. XscFv configurations 1-4 without linkers are labeled 1-4, respectively.

FIGS. 3A and 3B depict a model in a standard scFv configuration. FIG. 3A shows a model in a standard scFv configuration in ribbon diagram format. FIG. 3B shows in a standard scFv configuration in two-dimensional ribbon diagram format. Light chain variable region beta sheets 1-9 are labeled A-G (red), respectively. Heavy chain variable regions beta sheets 1-9 are labeled A-G (blue), respectively.

FIGS. 4A and 4B depict a model in xscFv configuration 1. FIG. 4A shows a model in xscFv configuration 1 in ribbon diagram format. FIG. 4B shows in xscFv configuration 1 in two-dimensional ribbon diagram format. Light chain variable region beta sheets 2-9 are labeled B-G (red), respectively. Heavy chain variable regions beta sheets 1-9 are labeled A-G (blue), respectively.

FIGS. 5A and 5B depict a model in xscFv configuration 2. FIG. 5A shows a model in xscFv configuration 2 in ribbon diagram format. FIG. 5B shows in xscFv configuration 2 in two-dimensional ribbon diagram format. Light chain variable region beta sheets 1-9 are labeled A-G (red), respectively. Heavy chain variable regions beta sheets 2-9 are labeled B-G (blue), respectively.

FIGS. 6A and 6B depict a model in xscFv configuration 3. FIG. 6A shows a model in xscFv configuration 3 in ribbon diagram format. FIG. 6B shows in xscFv configuration 3 in two-dimensional ribbon diagram format. Light chain variable region beta sheets 2-9 are labeled B-G (red), respectively. Heavy chain variable regions beta sheets 2-9 are labeled B-G (blue), respectively.

FIGS. 7A and 7B depict a model in xscFv configuration 4. FIG. 7A shows a model in xscFv configuration 4 in ribbon diagram format. FIG. 7B shows in xscFv configuration 4 in two-dimensional ribbon diagram format. Light chain variable region beta sheets 2-9 are labeled B-G (red), respectively. Heavy chain variable regions beta sheets 2-9 are labeled B-G (blue), respectively.

FIG. 8 depicts a superposition of a standard scFv model (V_{H}=dark blue and V_{L}=red) with an xscFv model (light blue and pink). scFv linker is depicted in yellow.

FIGS. 9A-9C depict superpositions of models of xscFv configurations. FIG. 9A depicts a superposition of xscFv configurations 1 (red) and 3 (green). The crossover points for xscFv configurations 1 and 3 are identified with red and green arrows, respectively. FIG. 9B depicts a superposition of xscFv configurations 2 (purple) and 4 (blue). The crossover points for xscFv configurations 2 and 4 are identified with purple and blue arrows, respectively. FIG. 9C depicts a superposition of xscFv configurations 1 (red) and 4 (blue). The crossover points for xscFv configurations 1 and 4 are identified with red and blue arrows, respectively.

FIG. 10 provides the heavy chain variable region (1N8Z:B|V_{H}, blue) and light chain variable region (1N8Z:A|V_{K}, red) amino acid sequences of a standard scFv, and the location of the same amino acid residues in xscFv configurations 1-4 (V_{H}=blue and V_{L}=red). The CDRs are underlined.

FIG. 11 depicts a superposition of models in space filling (cpk) format of a standard scFv (V_{L} (red), V_{H} (green), linker (yellow)) and xscFv configuration 4 (blue). The two molecules superimpose almost identically in shape. Minor differences in atomic positions are standard for two independently minimized structures.

FIG. 12 discloses a protein A chromatography purification of xscFv clone cell culture supernatants and purified trastuzumab xscFvs on two non-reducing SDS-PAGE gels. The gel columns are labeled as follows: “Sup” contains the raw supernatant, “W 1” contains the wash, “E” contains the low pH elution, “Fn1” contains the final purified xscFv (buffer exchanged and concentrated to ˜0.1 mg/ml), and “Hertn” contains the Herceptin control (at ˜0.3 mg/ml). Columns 2-5 of gel 1 were obtained from xscFv configuration 1 cell cultures. Columns 6-9 of gel 1 were obtained from xscFv configuration 2 cell cultures. Columns 2-5 of gel 2 were obtained from xscFv configuration 3 cell cultures. Columns 6-9 of gel 2 were obtained from xscFv configuration 4 cell cultures.

FIG. 13 shows data from a plate-based ELISA for four trastuzumab xseFv-Fe designs (shades of blue) and controls. Negative controls include full-length therapeutic mAbs and scFv-Fcs that bind other targets as well as a mix of human isotype IgG's (shades of red). Positive control trastuzumab scFv-Fc is shown in green.

FIG. 14 shows a 2-dimensional IMGT Collier de Perles numbered depiction of trastuzumab VL (i.e., V-Kappa domain). The sequences for each of the A_{L}, B_{L}, C_{L}, C′_{L}, C″_{L}, D_{L}, E_{L}, F_{L}, and G_{L} (SEQ ID NOS: 1-8 and 10, respectively) are shown above the corresponding letters (i.e., A, B, C, C′, C″, D, E, F, and G). These sequences correspond to the A_{L}, B_{L}, C_{L}, C′_{L}, C″_{L}, D_{L}, E_{L}, F_{L}, and G_{L} beta sheet ribbon diagrams depicted in the preceding drawings. Hatched circles indicate missing positions according to the IMGT unique numbering.

FIG. 15 shows the polypeptide sequence in each of the A_{L}, B_{L}, C_{L}, C′_{L}, C″_{L}, D_{L}, E_{L}, F_{L}, and G_{L} segments from the VL domain as utilized in generating a trastuzumab-based xscFv as described herein.

FIG. 16 shows a 2-dimensional IMGT Collier de Perles numbered depiction of trastuzumab VH domain. The sequences for each of the A_{H}, B_{H}, C_{H}, C′_{H}, C″_{H}, D_{H}, E_{H}, F_{H}, and G_{H} (SEQ ID NOS:11-18 and 20, respectively) are shown above the corresponding letters (i.e., A, B, C, C′, C″, D, E, F, and G). These sequences correspond to the A_{H}, B_{H}, C_{H}, C′_{H}, C″_{H}, D_{H}, E_{H}, F_{H}, and G_{H} beta sheet ribbon diagrams depicted in the preceding drawings. Hatched circles indicate missing positions according to the IMGT unique numbering.

FIG. 17 shows the polypeptide sequence in each of the A_{H}, B_{H}, C_{H}, C′_{H}, C″_{H}, D_{H}, E_{H}, F_{H}, and G_{H} segments from the VH domain as utilized in generating a trastuzumab-based xscFv as described herein.

FIG. 18 shows the amino acid sequence of xscFv_1-Fc_fusion (SEQ ID NO:50), which is an exemplary xscFv-Fc fusion protein. The first block of shaded text (“SP”), from amino acid positions 1 to 20, indicates the predicted signal peptide sequence. The mature xscFv_1-Fc (residues 21-467 of SEQ ID NO:50) is not expected to include the signal peptide sequence. “Disulfide” and highlighted cysteine residues “C”, at amino acid positions 42, 109, 139, and 211, indicate amino acid positions where predicted disulfide bonds can occur. The shaded “Hinge, Fe” C-terminal region, from amino acid positions 236 to 467, indicates the peptide hinge and Fc region as fused to the end of the xscFv polypeptide. See also, Table 5.

FIG. 19 shows the amino acid sequence of xscFv_2-Fc_fusion (SEQ ID NO:51), which is an exemplary xscFv-Fc fusion protein. The first block of shaded text (“SP”), from amino acid positions 1 to 20, indicates the predicted signal peptide sequence. The mature xscFv 2-Fc (residues 21-468 of SEQ ID NO:51) is not expected to include the signal peptide sequence. “Disulfide” and highlighted cysteine residues “C”, at amino acid positions 43, 115, 149, and 216, indicate amino acid positions where predicted disulfide bonds can occur. The shaded “Hinge, Fe” C-terminal region, from amino acid positions 237 to 468, indicates the peptide hinge and Fe region as fused to the end of the xscFv polypeptide. See also, Table 5.

FIG. 20 shows the amino acid sequence of xscFv_3-Fc_fusion (SEQ ID NO:52), which is an exemplary xscFv-Fc fusion protein. The first block of shaded text (“SP”), from amino acid positions 1 to 20, indicates the predicted signal peptide sequence. The mature xscFv 3-Fe (residues 21-455 of SEQ ID NO:52) is not expected to include the signal peptide sequence. “Disulfide” and highlighted cysteine residues “C”, at amino acid positions 68, 98, 170, and 203, indicate amino acid positions where predicted disulfide bonds can occur. The shaded “Hinge, Fe” C-terminal region, from amino acid positions 224 to 455, indicates the peptide hinge and Fc region as fused to the end of the xscFv polypeptide. See also, Table 5.

FIG. 21 shows the amino acid sequence of xscFv_4-Fc_fusion (SEQ ID NO:53), which is an exemplary xscFv-Fc fusion protein. The first block of shaded text (“SP”), from amino acid positions 1 to 20, indicates the predicted signal peptide sequence. The mature xscFv 4-Fc (residues 21-455 of SEQ ID NO:53) is not expected to include the signal peptide sequence. “Disulfide” and highlighted cysteine residues “C”, at amino acid positions 75, 109, 176, and 206, indicate amino acid positions where predicted disulfide bonds can occur. The shaded “Hinge, Fe” C-terminal region, from amino acid positions 224 to 455, indicates the peptide hinge and Fc region as fused to the end of the xscFv polypeptide. See also, Table 5.

### DETAILED DESCRIPTION

The antibody polypeptide comprising crossover scFvs with a single light chain variable domain and a single heavy chain variable domain are provided. In one aspect, the xscFv does not comprise a linker. The antibody polypeptide may also comprise additional domains, such as an Fc domain or additional V_{H}s and V_{L}s. The antibody polypeptides are less immunogenic, more stable and less likely to aggregate than standard scFvs. The antibody polypeptides are useful in any applications where scFvs may otherwise be used.

Accordingly, in one aspect, the antibody polypeptide comprises an xscFv, wherein the V_{L} and V_{H} beta strands are positioned one of the single polypeptide chain conformations as follows:
xscFv 1: A_{H}→B_{H}→C_{H}→C′_{L}→C″_{L}→D_{L}→E_{L}→F_{L}→G_{L}→B_{L}→C_{L}→C′_{H}→C″_{H}→D_{H}→E_{H}→F_{H}→G_{H}
xscFv 2: A_{L}→B_{L}→C_{L}→C′_{H}→C″_{H}→D_{H}→E_{H}→F_{H}→G_{H}→B_{H}→C_{H}→C′_{L}→E_{L}→F_{L}→G_{L}
xscFv 3: C′_{L}→C″_{L}→D_{L}→E_{L}→F_{L}→G_{L}→B_{L}→C_{L}→C′_{H}→C″_{H}→D_{H}→E_{H}→F_{H}→G_{H}→B_{H}→C_{H}
xscFv 4: C′_{H}→C″_{H}→D_{H}→E_{H}→F_{H}→G_{H}→B_{H}→C_{H}→C′_{L}→C″_{L}→D_{L}→E_{L}→F_{L}→G_{L}→B_{L}→C_{L}

The antibody polypeptides with specific binding properties may be selected using a primary screen that utilizes antigen and cell binding assays, followed by one or more rounds of error-prone or degenerate oligonucleotide-directed affinity maturation. As a result, a genus of xscFvs with varying variable domain sequences are provided.

As used herein, “specific binding” refers to the binding of an antigen by an antibody polypeptide with a dissociation constant (K_{d}) of about 1 μM or lower as measured, for example, by surface plasmon resonance (SPR). Suitable assay systems include the BIAcore™ surface plasmon resonance system and BIAcore™ kinetic evaluation software (e.g., version 2.1). The affinity or K_{d} for a specific binding interaction may be about 1 micromolar (μM) or lower, about 500 nanomolar (nM) or lower, about 300 nM or lower, about 100 nM or lower, about 50 nM or lower, about 20 nM or lower, about 10 nM or lower, or about 1 nM or lower.

Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in PBS (phosphate buffered saline) at pH 7.2 at 30° C. These techniques can be used to measure the concentration of bound and free binding protein as a function of binding protein (or target) concentration. The concentration of bound binding protein ([Bound]) is related to the concentration of free binding protein ([Free]) and the concentration of binding sites for the binding protein on the target where (N) is the number of binding sites per target molecule by the following equation:

[Bound]=N·[Free]/((1/Ka)+[Free]).

It is not always necessary to make an exact determination of K_{D}, though, since sometimes it is sufficient to obtain a quantitative measurement of affinity, e.g., determined using a method such as ELISA or FACS analysis, is proportional to K_{D}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

The term “about” will be understood by persons of ordinary skill in the art and will vary to some extent on the context in which it is used. Generally, about encompasses a range of values that are plus/minus 10% of a referenced value.

In accordance with this detailed description, the following abbreviations and definitions apply. It must be noted that as used herein, the singular forms “a,” “an,” and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

1. Antibody Polypeptide Functional Domains

The antibody polypeptides comprise two variable domains. In one embodiment, the antibody polypeptides are in the form of a crossover single chain variable fragment (xscFv) capable of specifically and monovalently binding an antigen. An xscFv comprises an intercalated V_{L} and V_{H} structure, i.e., comprises interdomain and/or intradomain intercalations (insertions) wherein at least one portion of a V_{L} domain (e.g., one or more beta-strands) or at least one portion of a VH domain (e.g., one or more beta-strands) is inserted within a different portion (or region) of the VL domain or of the VH domain (including vice versa), while retaining antigen binding capacity/ability. A crossover single chain variable fragment is also referred to herein as an intercalated single chain variable fragment.

As used herein, the term “variable domain” refers to immunoglobulin variable domains defined by Kabat et al., Sequences of Immunological Interest, 5^{th} ed., U.S. Dept. Health & Human Services, Washington, D.C. (1991). The variable domains (V_{L} or V_{H}) are about 100-120 amino acids each. The variable domain has several regions, some of which are more conserved than others. Analysis of the antibody coding sequences has shown two classes of variable regions within the Fv, hypervariable sequences (or complementarity determining regions (CDRs)) and framework sequences. The numbering and positioning of CDR amino acid residues within the variable domains is in accordance with the well-known Kabat numbering convention. The complementarity determining regions (CDRs) contained therein are primarily responsible for antigen recognition, although framework residues can play a role in epitope binding.

Light chains are classified as kappa (κ) or lambda (λ), and are characterized by a particular constant region, CL, as known in the art. Heavy chains are classified as y, a, 6, or c, and define the isotype of an antibody as IgG, IgM, IgA, IgD, or IgE, respectively. The heavy chain constant region is comprised of three domains, CH1, CH2, and CH3, for IgG, IgD, and IgA; and four domains, CH1, CH2, CH3, and CH4, for IgM and IgE.

Each light chain variable domain (V_{L}) and heavy chain variable domain (V_{H}) is composed of three CDRs and four framework regions (FRs), arranged from amino-terminus to carboxy-terminus in the following order: FR1, CDR1, FR2, CDR2, FR3, CDR3, and FR4. The three CDRs of the light chain can be referred to as LCDR1, LCDR2, and LCDR3 and the three CDRs of the heavy chain can be referred to as HCDR1, HCDR2, and HCDR3.

Variable domains may comprise one or more FR with the same amino acid sequence as a corresponding framework region encoded by a human germline antibody gene segment. For example, an antibody polypeptide may comprise the V_{H} germline gene segments DP47, DP45, or DP38, the V_{κ} (V-kappa light chain) germline gene segment DPK9, the J_{H} (J-heavy chain) segment JH4b, or the J_{κ} (J-kappa light chain) segment.

Antibody polypeptides also may be “fragments” comprising a portion of a full-length immunoglobulin molecule that comprises intercalated variable domains. Thus, the term “antibody polypeptide” includes a IgG AC_{H}2, a single chain Fv (scFv), scFv-Fc, (scFv)₂, Fab fragment, F(ab′)₂ fragment, F(ab′)₃ fragment, Fv fragment, dsFv, diabody, triabody, tetrabody, minibody, bispecific antibody, or bispecific single-chain Fvs (bsscFvs), for example, wherein heavy chain and light chain variable regions are intercalated (inserted) as described herein. The term “antibody polypeptides” thus includes polypeptides made by recombinant engineering and expression.

Antibody polypeptides comprise two variable domains. Prior to the present invention, the “traditional,” “classic,” or “standard” scFv configuration has been one wherein the C-terminus of a heavy chain variable domain is connected by a linker sequence to the N-terminus of a light chain variable domain (which may be written herein as: VH→linker→VL, or simply VH-VL). Conversely, scFv have also been configured the other way around, wherein the C-terminus of a light chain variable domain is connected by a linker sequence to the N-terminus of a heavy chain variable domain (which may be written herein as: VL-->linker-->VH, or simply VL-VH).

In contrast to standard scFv configurations, in one embodiment antibody polypeptides are in the form of a crossover (“x”) single chain variable fragment (referred to herein as “xscFv”). An xscFv comprises an intercalated VL and VH configuration (comprising interdomain and/or intradomain intercalations (insertions)) and is capable of specifically and monovalently binding an antigen.

Polypeptides such as antibodies (i.e., immunoglobulins) have a primary, secondary, tertiary, and quaternary structure. The primary structure is the linear amino acid sequence of the polypeptide chains. The secondary structure is the three-dimensional form a polypeptide has over local segments (small stretches) of the polypeptide; these are most recognizable as alpha helices, beta sheets, loops and coiled regions. Protein tertiary structure is the overall geometric shape or architectural configuration of a polypeptide, which results from the assemblage of combined primary and secondary structures. Quaternary structure is the arrangement and overall geometric shape (architecture) of a multi-subunit complex of two or more polypeptides (such as the assemblage of two VH-CH polypeptides and two VL-CL polypeptides into a full length native antibody in vivo).

The intercalated or crossover scFv (xscFv) may make use, or take advantage, of the tertiary and quaternary structure of immunoglobulin VH and VL domains by introducing one or more peptide bonds (“engineered peptide bond”) to rearrange (reconnect) the linear sequence (primary structure) of the variable domains while maintaining the overall tertiary and/or quaternary structure of complexed VH and VL antibody binding domains (or the tertiary structure of combined VH-VL (or VL-VH) domains in the case of traditional scFv). Accordingly, the intercalated (inserted) polypeptide sequences, which form xscFv, function to retain antigen binding ability while reducing overall VH-VL linker length. xscFv are designed and constructed by observing and making use of predicted or known antibody tertiary and/or quaternary structure (e.g., crystal structures) and selecting suitable “crossover” points in the overall architecture to identify portions of the tertiary structure which come into close proximity with each other, approximately 7-14, 8-13 or 9-12 Å, and where both proximate portions (e.g., “loops”) have compatible N→C/N→C or C←N/C←N directionality for relocation of peptide bonds, even though these portions may be otherwise widely or significantly separated from each other as compared to the linear sequence (primary structure) of the variable domains found in “traditional” antibodies (scFv VH-VL or full-length VH-CH/VL-CL). Thus, a “crossover” as defined herein allows the linear sequence of the variable domains in an scFv (or other any other type of VH/VL antibody tertiary and quaternary structure) to be significantly rearranged by relocating suitable peptide bonds as identified in the tertiary and/or quaternary structure (known or predicted) of the associated VH/VL domains (e.g., VL-VH or VL-VH scFvs) such that new peptide bonds are generated to crossover in the midst of the VH and/or VL linear sequences without disturbing (or without substantially disturbing) the overall tertiary or quaternary structure of the VH/VL (e.g., scFV VH-VL or VL-VH) immunoglobulin architecture (and hence without affecting, or without substantially affecting antigen binding ability). The crossovers thus eliminate the need for noncovalent bonds, linkers, or other antibody frameworks to hold the VH/VL domains together in an immunoglobulin fold.

While not limited by any particular theory, it is believed that the xscFv disclosed do not aggregate, because the lack of long peptide linkers prevent domain cross-binding events, where the V domain of one chain interacts with a corresponding V domain from a second chain. The xscFv, lacking long linkers and not having two independently folding V domains, would be unable to aggregate by this mechanism.

Not only can crossovers occur between antibody domains (interdomain crossover), but they can be made within antibody domains (intradomain crossover). Notably, intradomain crossovers differ from interdomain crossovers in that intradomain crossovers bridge non-consecutive beta strands within a domain. Both types of crossovers must maintain the above-described spatial proximity and compatible N-->C sequence directionality.

The carboxy-terminal “half” of each heavy chain in a standard immunoglobulin configuration defines a constant region (Fe) primarily responsible for effector function. As used herein, the term “Fe domain” refers to the constant region antibody sequences comprising CH2 and CH3 constant domains as delimited according to Kabat et al., Sequences of Immunological Interest, 5^{th} ed., U.S. Dept. Health & Human Services, Washington, D.C. (1991). The Fc domain may be derived from an IgG1 or an IgG4 Fc region, for example. A variable domain may be fused to an Fc domain.

When a variable domain of an xscFv is fused to an Fc domain, the carboxyl terminus of the intercalated variable domain (either a V_{L} or V_{H} domain) may be linked or fused to the amino terminus of the Fc CH2 domain. Alternatively, the carboxyl terminus of the variable domain may be linked or fused to the amino terminus of a CH1 domain, which itself is fused to the Fc CH2 domain. The protein may comprise the hinge region between the CH1 and CH2 domains in whole or in part.

An “epitope” refers to the site on a target compound that is bound by an antibody polypeptide. In the case where the target compound is a protein, the site can be entirely composed of amino acid components, entirely composed of chemical modifications of amino acids of the protein (e.g., glycosyl moieties), or composed of combinations thereof. Overlapping epitopes include at least one common amino acid residue.

The term “human,” when applied to antibody polypeptides, means that the antibody polypeptide has a sequence, e.g., framework regions and/or CH domains, derived from a human immunoglobulin. A sequence is “derived from” a human immunoglobulin coding sequence when the sequence is either: (a) isolated from a human individual or from a cell or cell line from a human individual; (b) isolated from a library of cloned human antibody gene sequences or of human antibody variable domain sequences; or (c) diversified by mutation and selection from one or more of the polypeptides above. An “isolated” compound as used herein means that the compound is removed from at least one component with which the compound is naturally associated with in nature.

Human antibody polypeptides can be administered to human patients while largely avoiding the anti-antibody immune response often provoked by the administration of antibodies from other species, e.g., mouse. For example, murine antibodies can be “humanized” by grafting murine CDRs onto a human variable domain FR, according to procedures well known in the art. Human antibodies as disclosed herein, however, can be produced without the need for genetic manipulation of a murine antibody sequence.

2. Antibody Polypeptide Primary, Secondary, and Tertiary Structure

Standard immunoglobulin domains are composed of between 7 (for constant domains) and 9 (for variable domains) beta strands (β-strands; also known as beta-sheets (β-sheets)). See “Antibody Structure” at www.bioatla.com/wp-content/uploads/Appendix_antibodystructure.pdf. Short beta-sheet peptides, which are coded by minigenes (framework regions), are linked together by random order peptides that are not coded by an original gene. The framework sequences are responsible for the correct beta-sheet folding of the variable domains, and also for the inter-chain interactions that bring both domains together. The immunoglobulin light chain variable domain is expressed as one continuous polypeptide chain, wherein the light chain beta sheets are separated by loops. The sequence of the beta sheets in the continuous polypeptide chain can be expressed as follows (with loops indicated with arrows):
A_{L}→B_{L}→C_{L}→C′_{L}→C″_{L}→D_{L}→E_{L}→F_{L}→G_{L}

The immunoglobulin heavy chain variable domain is expressed as one continuous polypeptide chain, wherein the heavy chain beta sheets are separated by loops. The sequence of the beta sheets in the continuous polypeptide chain can be expressed as follows:
A_{H}→B_{H}→C_{H}→C′_{H}→C″_{H}→D_{H}→E_{H}→F_{H}→G_{H}

Each domain (both variable and constant) in an immunoglobulin has a similar structure of two beta sheets packed tightly against each other in a conserved, sandwiched, compressed, anti-parallel beta barrel termed “the immunoglobulin fold.” The “sandwich” shape is held together by interactions between conserved cysteines and other charged amino acids. The folds of variable domains have 9 beta strands arranged in two sheets of 4 and 5 strands. The 5-stranded sheet is packed against the 4-stranded sheet. The fold is stabilized by hydrogen bonding between the beta strands of each sheet, by hydrophobic bonding between residues of opposite sheets in the interior, and by a disulfide bond between the sheets. In each variable domain, the 5-stranded sheet comprises strands C, F, G, C′ and C″ and the 4-stranded sheet has strands A, B, E, and D. A disulfide bond links strands B and F in opposite sheets. See “An Introduction to Immunoglobulin Structure” at http://www.callutheran.edu/BioDev/omm/ig/molmast.htm.

In an scFv, the light chain variable domains and heavy chain variable domains are expressed in one continuous polypeptide chain, wherein the C terminus of the V_{L} is connected to the N terminus of the V_{H} by a linker, or visa versa. The location of the immunoglobulin beta strands in the continuous polypeptide chain can be expressed as follows:
A_{L}→B_{L}→C_{L}→C′_{L}→C″_{L}→D_{L}→E_{L}→F_{L}→G_{L}→[a linker]→A_{H}→B_{H}→C_{H}→C′_{H}→C″_{H}→D_{H}→E_{H}→F_{H}→G_{H}

Much like a full-length antibody, the short beta-sheet peptides (framework regions), are responsible for the immunoglobulin-like beta-sheet folding of the variable domains, and also for the inter-chain interactions that bring both domains together in an scFv configuration. The folds of variable domains have 9 beta strands arranged in two sheets of 4 and 5 strands. The 5-stranded sheet is packed against the 4-stranded sheet. In each variable domain, the 5-stranded sheet comprises strands C, F, G, C′ and C″ and the 4-stranded sheet has strands A, B, E, and D. A disulfide bond links strands B and F in opposite sheets.

Crossover scFvs (xscFvs) are constructed by intercalating (i.e., inserting or relocating) portions (e.g., beta strands) of heavy and light chains of an scFv at specific points in the folded protein based on the topology of the V domains. These points allow for crossovers between the two variable domains of an scFv which produces a single chain Fv that conserves the Ig (immunoglobulin) folds of both the V_{H} and the V_{L} domains when expressed and folded. Unlike scFvs, xscFvs do not require a linker to sequentially connect the V_{H} and V_{L} chains, because of their intercalated fold. In an xscFv, the light chain variable domains and heavy chain variable domains are expressed in one continuous polypeptide chain, however the sequence of the 13 strands differs from the sequence of the β strands in full antibodies, Fabs and scFvs. In an xscFv, at least one portion of the heavy chain variable domain or the light chain variable domain is relocated to another location in the scFv when compared to a standard scFv.

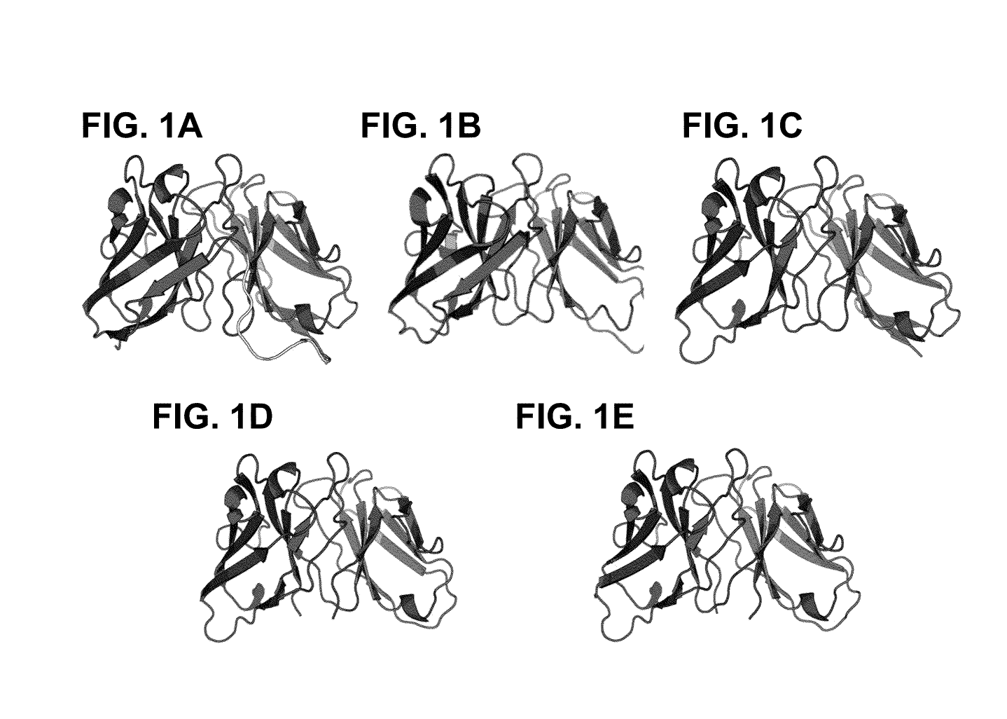

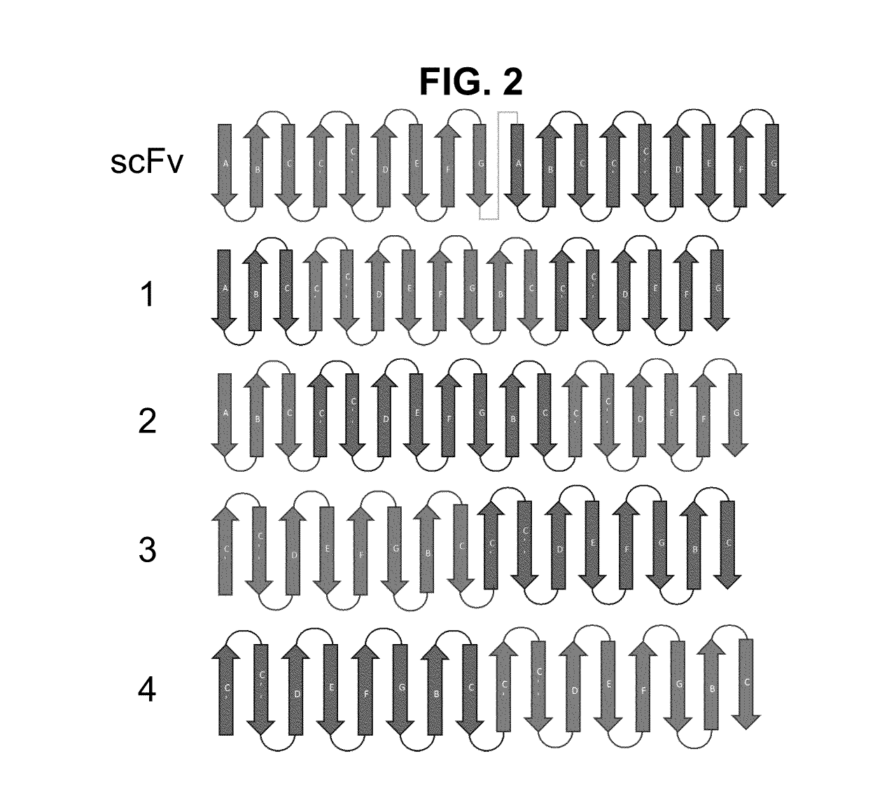

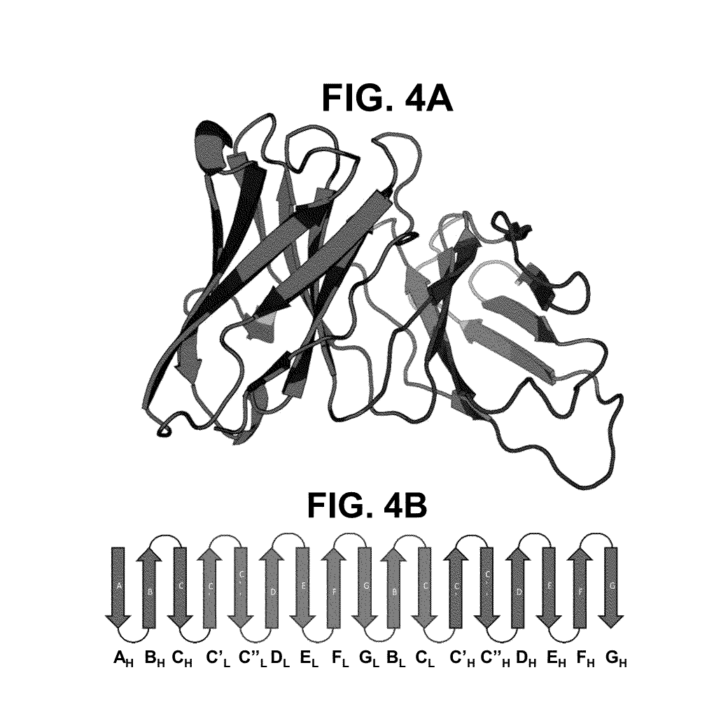

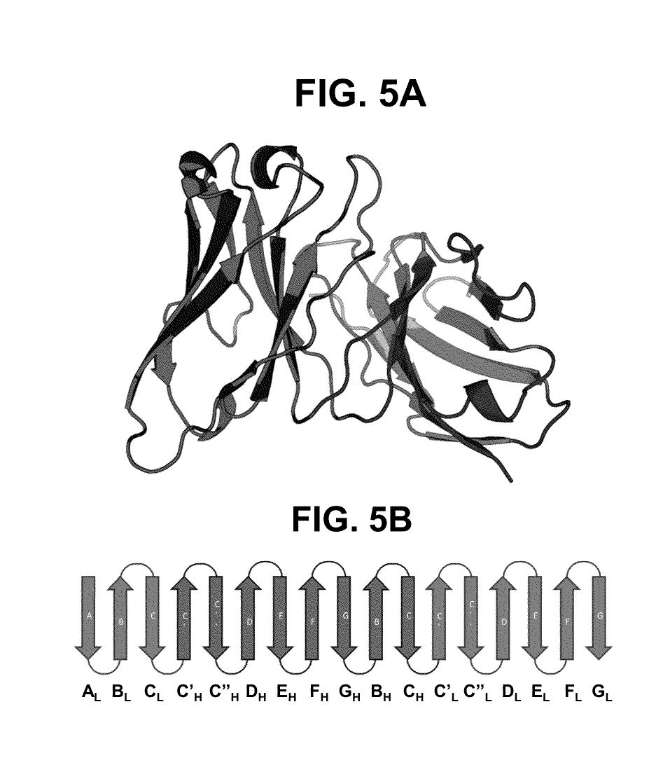

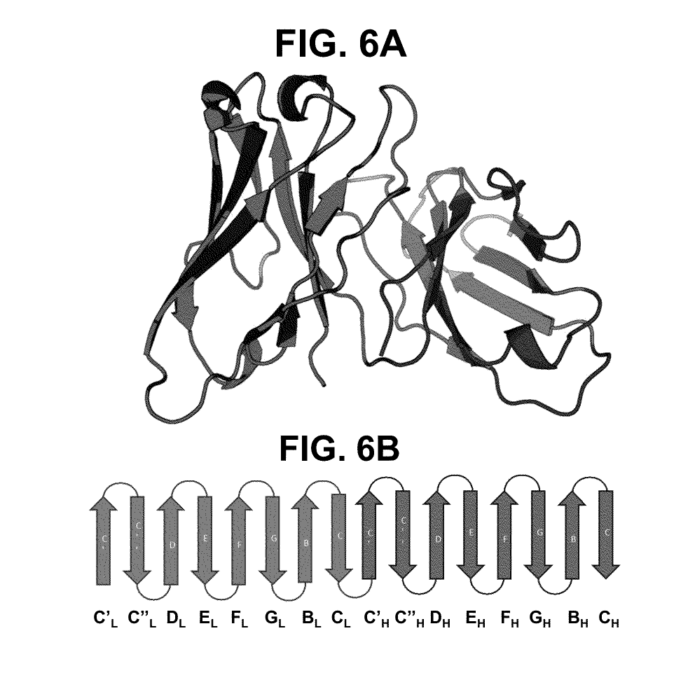

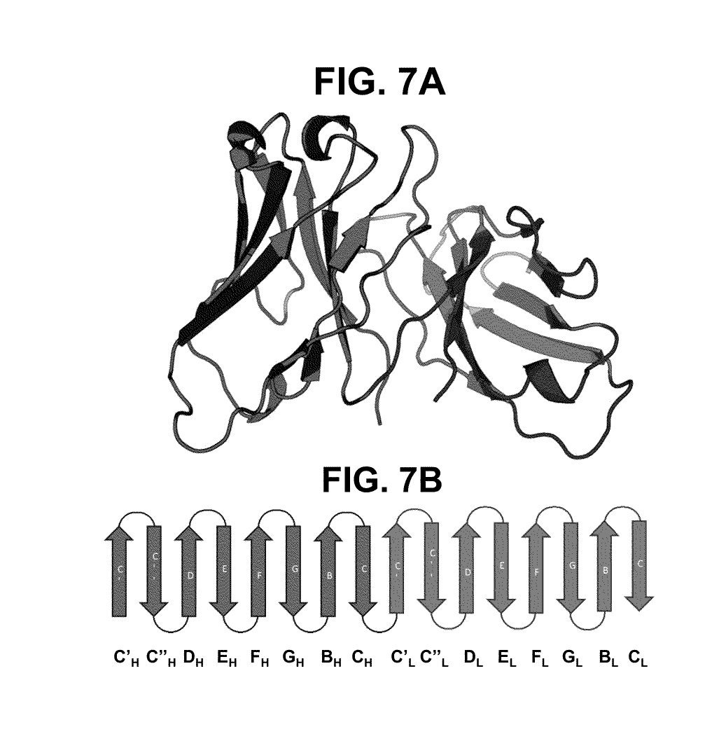

The location of the immunoglobulin beta strands in an xscFv polypeptide chain is exemplified in the four examples below (see also FIGS. 1B-1E, 2, 4A, 4B, 5A, 5B, 6A, 6B, 7A, and 7B):

xscFv 1: A_{H}→B_{H}→C_{H}→C′_{L}→C″_{L}-D_{L}→E_{L}→F_{L}→G_{L}→B_{L}→C_{L}→C′_{H}→C″_{H}→D_{H}→E_{H}→F_{H}→G_{H}
xscFv 2: A_{L}→B_{L}→C_{L}→C′_{H}→C″_{H}→D_{H}→E_{H}→F_{H}→G_{H}→B_{H}→C_{H}→C′_{L}→C″_{L}→D_{L}→E_{L}→F_{L}→G_{L}
xscFv 3: C′_{L}→C″_{L}→D_{L}→E_{L}→F_{L}→G_{L}→B_{L}→C_{L}→C′_{H}→C″_{H}→D_{H}→E_{H}→F_{H}→G_{H}→B_{H}→C_{H}
xscFv 4: C_{H}→C″_{H}→D_{H}→E_{H}→F_{H}→G_{H}→B_{H}→C_{H}→C′_{L}→C″_{L}→D_{L}→E_{L}→F_{L}→G_{L}→B_{L}→C_{L}

The three heavy chain and three light chain CDRs comprise six total hypervariable loops. The six hypervariable loops of both chains form the antigen binding site. The residues in the CDRs vary from one immunoglobulin molecule to the next, imparting antigen specificity to each antibody. See “An Introduction to Immunoglobulin Structure” at http://www.callutheran.edu/BioDev/omm/ig/molmast.htm. The hypervariable loops are connected by beta strands B-C, C′-C″, and F-G of the immunoglobulin fold. The three CDRs of the V_{L} or V_{H} domain (CDR1, CDR2, CDR3) cluster at one end of the beta barrel. The V_{L} and V_{H} domains at the tips of antibody molecules are closely packed such that the 6 CDRs cooperate in constructing a surface for antigen-specific binding. Residues in all six CDRs (V_{L} CDR1, CDR2, CDR3 and V_{H} CDR1, CDR2, CDR3) project from the distal surface of the antibody tip, in position to recognize and bind antigen. See “An Introduction to Immunoglobulin Structure” at http://www.callutheran.edu/BioDev/omm/ig/molmast.htm.

3. Antibody Polypeptide Sequence Selection

Changes may be made to antibody polypeptide sequences while retaining antigen binding specificity. Error-prone affinity maturation provides one exemplary method for making and identifying antibody polypeptides with variant sequences that retain the specificity of the original antibody polypeptide.

In one embodiment, amino acid substitutions may be made to individual FR regions, such that an FR comprises 1, 2, 3, 4, or 5 amino acid differences relative to the amino acid sequence of the corresponding FR encoded by a human germline antibody gene segment. In another embodiment, the variant variable domain may contain one or two amino acid substitutions in a CDR. In other embodiments, amino acid substitutions to FR and CDR regions may be combined.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). It is possible for many framework and CDR amino acid residues to include one or more conservative substitutions.

Consensus sequences for antibody polypeptides can include positions which can be varied among various amino acids. For example, the symbol “X” in such a context generally refers to any amino acid (e.g., any of the twenty natural amino acids or any of the nineteen non-cysteine amino acids). Other allowed amino acids can also be indicated for example, using parentheses and slashes. For example, “(A/W/F/N/Q)” means that alanine, tryptophan, phenylalanine, asparagine, and glutamine are allowed at that particular position.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blosum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

In one embodiment, the length of a reference sequence aligned for comparison purposes is at least 30%, at least 40%, at least 50%, at least 60%, at least 70%, 80%, 90%, 92%, 95%, 97%, 98%, or 100% of the length of the reference sequence. For example, the reference sequence may be the length of the immunoglobulin variable domain sequence.

An antibody polypeptide may have mutations (e.g., at least one, two, or four, and/or less than 15, 10, 5, or 3) relative to a binding protein described herein (e.g., a conservative or non-essential amino acid substitutions), which do not have a substantial effect on the protein functions. Whether or not a particular substitution will be tolerated, i.e., will not adversely affect biological properties, such as binding activity can be predicted, e.g., using the method of Bowie, et al. (1990) Science 247:1306-1310.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and non-aqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and (4) very high stringency hybridization conditions are 0.5M sodium phosphate, 7% SDS at 65° C., followed by one or more washes at 0.2×SSC, 1% SDS at, 65° C. Very high stringency conditions (4) are the preferred conditions and the ones that should be used unless otherwise specified. The disclosure includes nucleic acids that hybridize with low, medium, high, or very high stringency to a nucleic acid described herein or to a complement thereof, e.g., nucleic acids encoding a binding protein described herein. The nucleic acids can be the same length or within 30, 20, or 10% of the length of the reference nucleic acid. The nucleic acid can correspond to a region encoding an immunoglobulin variable domain sequence.

As used herein, the term “substantially identical” (or “substantially homologous”) is used herein to refer to a first amino acid or nucleic acid sequence that contains a sufficient number of identical or equivalent (e.g., with a similar side chain, e.g., conserved amino acid substitutions) amino acid residues or nucleotides to a second amino acid or nucleic acid sequence such that the first and second amino acid or nucleic acid sequences have (or encode proteins having) similar activities, e.g., a binding activity, a binding preference, or a biological activity. In the case of antibodies, the second antibody has the same specificity and has at least 50% of the affinity relative to the same antigen.

Sequences similar or homologous (e.g., at least about 85% sequence identity) to any sequences disclosed herein are also part of this application. In some embodiments, the sequence identity can be about 85%, 90%, 91%, 92%, 93%, 94%, 95%, 96%, 97%, 98%, 99% or higher. In addition, substantial identity exists when the nucleic acid segments hybridize under selective hybridization conditions (e.g., highly stringent hybridization conditions), to the complement of the strand. The nucleic acids may be present in whole cells, in a cell lysate, or in a partially purified or substantially pure form.

Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02. Particular binding proteins may show a difference, e.g., in specificity or binding, that are statistically significant (e.g., P value <0.05 or 0.02). The terms “induce,” “inhibit,” “potentiate,” “elevate” “increase,” “decrease,” or the like, e.g., which denote distinguishable qualitative or quantitative differences between two states, and may refer to a difference, e.g., a statistically significant difference, between the two states.

Trastuzumab VH and VL sequence information was used to design exemplary xscFvs. The xscFv gene with NheI and BamH I restriction sites was gene synthesized for cloning into CMV epiPuro™ vector. Based on the disclosed amino acid and polynucleotide sequences, the fusion protein was produced in 293T cells. The xscFv-Fc protein was purified using protein A affinity chromatography. The information regarding the boundaries of the V_{L} or V_{H} domains of heavy and light chain genes may be used to design PCR primers to amplify the variable domain from a cloned heavy or light chain coding sequence encoding an antibody polypeptide known to bind a specific antigen. The amplified variable domain may be inserted into a suitable expression vector, e.g., pHEN-1 (Hoogenboom et al. (1991) Nucleic Acids Res. 19:4133-4137) and expressed, either alone or as a fusion with another polypeptide sequence, using techniques well known in the art. Based on the disclosed amino acid and polynucleotide sequences, the fusion protein can be produced and purified using ordinary skill in any suitable mammalian host cell line, such as CHO, 293, COS, NSO, and the like, followed by purification using one or a combination of methods, including protein A affinity chromatography, ion exchange, reverse phase techniques, or the like.

4. Further Antibody Polypeptide Formatting

In one aspect, the antibody polypeptide is a “dual specific” antibody polypeptide that binds two different antigens. In one embodiment, antibody polypeptides of a dual specific ligand may be linked by an “amino acid linker” or “linker.” For example, an xscFv may be fused to the N-terminus of an amino acid linker, and another xscFv may be fused to the C-terminus of the linker. Although amino acid linkers can be any length and consist of any combination of amino acids, the linker length may be relatively short (e.g., five or fewer amino acids) to reduce interactions between the linked domains. The amino acid composition of the linker also may be adjusted to reduce the number of amino acids with bulky side chains or amino acids likely to introduce secondary structure. Suitable amino acid linkers include, but are not limited to, those up to 3, 4, 5, 6, 7, 10, 15, 20, or 25 amino acids in length. Representative amino acid linker sequences include (GGGGS)ₙ, where n may be any integer between 1 and 5 (SEQ ID NOS:21-25 respectively). Other suitable linker sequences may be selected from the group consisting of AS, AST, TVAAPS (SEQ ID NO:26), TVA, and ASTSGPS (SEQ ID NO:27).

The binding of the second antigen can increase the in vivo half-life of the antibody polypeptide. For example, the second variable domain of the dual specific antibody polypeptide may specifically bind serum albumin (SA), e.g., human serum albumin (HSA). The antibody polypeptide formatted to bind I can have an increased in vivo t-α (“alpha half-life”) or t-β (“beta half-life”) half-life relative to the same unformatted antibody polypeptide. The t-α and t-β half-lives measure how quickly a substance is distributed in and eliminated from the body. The linkage to I may be accomplished by fusion of the antibody polypeptide with a second variable domain capable of specifically binding I, for example. Anti-human serum albumin antibodies are well-known in the art. See, e.g., Abeam®, Human Serum Albumin antibodies ab10241, ab2406, and ab8940, available on the Internet at hypertext transfer protocol www.abcam.com/index.html, or GenWay, ALB antibody, available on the Internet at hypertext transfer protocol www.genwaybio.com. Variable domains that specifically bind I can be obtained from any of these antibodies, and then fused to an antibody polypeptide of the disclosure using recombinant techniques that are well known in the art.

In another embodiment, an antibody polypeptide may be formatted to increase its in vivo half-life by PEGylation. In one embodiment, the PEG is covalently linked. In another embodiment, the PEG is linked to the antibody polypeptide at a cysteine or lysine residue. PEGylation can be achieved using several PEG attachment moieties including, but not limited to N-hydroxylsuccinimide active ester, succinimidyl propionate, maleimide, vinyl sulfone, or thiol. A PEG polymer can be linked to an antibody polypeptide at either a predetermined position, or can be randomly linked to the domain antibody molecule. PEGylation can also be mediated through a peptide linker attached to a domain antibody. That is, the PEG moiety can be attached to a peptide linker fused to an antibody polypeptide, where the linker provides the site (e.g., a free cysteine or lysine) for PEG attachment. Methods of PEGylating antibodies are well known in the art, as disclosed in Chapman, et al., “PEGylated antibodies and antibody fragments for improved therapy: a review,” Adv. Drug Deliv. Rev. 54(4):531-45 (2002), for example.

Antibody polypeptides also may be designed to form a dimer, trimer, tetramer, or other multimer. Antibody polypeptides such as xscFvs can be linked to form a multimer by several methods known in the art, including, but not limited to, expression of monomers as a fusion protein, linkage of two or more monomers via a peptide linker between monomers, or by chemically joining monomers after translation, either to each other directly, or through a linker by disulfide bonds, or by linkage to a di-, tri- or multivalent linking moiety (e.g., a multi-arm PEG).

5. Humanized Antibody Polypeptide Display Libraries

A display library can be used to identify antibody polypeptides, such as xscFvs, that bind to a specific antigen. A display library is a collection of entities; each entity includes an accessible polypeptide component and a recoverable component that encodes or identifies the polypeptide component. The polypeptide component is varied so that different amino acid sequences are represented. The polypeptide component can be of any length, e.g. from three amino acids to over 300 amino acids. In a selection, the polypeptide component of each member of the library is probed with the antigen of interest and if the polypeptide component binds to the antigen, the display library member is identified, typically by retention on a support. In addition, a display library entity can include more than one polypeptide component, for example, the two polypeptide chains of an sFab.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

Display libraries can include synthetic and/or natural diversity. See, e.g., US 2004-0005709. A variety of formats can be used for display libraries. Examples include the following:

Phage Display.

One format utilizes viruses, particularly bacteriophages. This format is termed “phage display.” The protein component is typically covalently linked to a bacteriophage coat protein. The linkage results from translation of a nucleic acid encoding the protein component fused to the coat protein. The linkage can include a flexible peptide linker, a protease site, or an amino acid incorporated as a result of suppression of a stop codon. Phage display is described, for example, in U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem. 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; and Hoogenboom et al. (1991) Nuc Acid Res 19:4133-4137.

Phage display systems have been developed for filamentous phage (phage fl, fd, and M13) as well as other bacteriophage. The filamentous phage display systems typically use fusions to a minor coat protein, such as gene III protein, and gene VIII protein, a major coat protein, but fusions to other coat proteins such as gene VI protein, gene VII protein, gene IX protein, or domains thereof can also been used (see, e.g., WO 00/71694). In one embodiment, the fusion is to a domain of the gene III protein, e.g., the anchor domain or “stump,” (see, e.g., U.S. Pat. No. 5,658,727 for a description of the gene III protein anchor domain). It is also possible to physically associate the protein being displayed to the coat using a non-peptide linkage.

Bacteriophage displaying the protein component can be grown and harvested using standard phage preparatory methods, e.g., PEG precipitation from growth media. After selection of individual display phages, the nucleic acid encoding the selected protein components can be isolated from cells infected with the selected phages or from the phage themselves, after amplification. Individual colonies or plaques can be picked, the nucleic acid isolated and sequenced.

Other Display Formats.

Other display formats include cell based display (see, e.g., WO 03/029456), protein-nucleic acid fusions (see, e.g., U.S. Pat. No. 6,207,446), and ribosome display (See, e.g., Mattheakis et al. (1994) Proc. Natl. Acad. Sci. USA 91:9022 and Hanes et al. (2000) Nat. Biotechnol. 18:1287-92; Hanes et al. (2000) Methods Enzymol. 328:404-30; and Schaffitzel et al. (1999)J Immunol Methods. 231 (1-2):119-35).

Display technology can also be used to obtain antibody polypeptides that bind particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library members that are not specific to the target.

Iterative Selection.

In one embodiment, display library technology is used in an iterative mode. A first display library is used to identify one or more antibody polypeptides that bind a target. These identified antibody polypeptides are then varied using a mutagenesis method to form a second display library. Higher affinity antibody polypeptides that are then selected from the second library, e.g., by using higher stringency or more competitive binding and washing conditions.

In some implementations, the mutagenesis is targeted to regions known or likely to be at the binding interface. In the case of antibody polypeptides, the mutagenesis can be directed to the CDR regions of the heavy or light chains as described herein. Further; mutagenesis can be directed to framework regions near or adjacent to the CDRs. In the case of antibody polypeptides, mutagenesis can also be limited to one or a few of the CDRs, e.g., to make precise step-wise improvements. Exemplary mutagenesis techniques include: error-prone PCR, recombination, DNA shuffling, site-directed mutagenesis and cassette mutagenesis.

In one example of iterative selection, the methods described herein are used to first identify an antibody polypeptide from a display library that binds an antigen of interest with at least a minimal binding specificity for a target or a minimal activity, e.g., an equilibrium dissociation constant for binding of less than 1 nM, 10 nM, or 100 nM. The nucleic acid sequence encoding the initial identified antibody polypeptides are used as a template nucleic acid for the introduction of variations, e.g., to identify a second antibody polypeptide that has enhanced properties (e.g., binding affinity, kinetics, or stability) relative to the initial antibody polypeptide.

Off-Rate Selection.

Since a slow dissociation rate can be predictive of high affinity, particularly with respect to interactions between antibody polypeptides and their targets, the methods described herein can be used to isolate antibody polypeptides with a desired kinetic dissociation rate (e.g., reduced) for a binding interaction to a target.

To select for slow dissociating antibody polypeptides from a display library, the library is contacted to an immobilized target. The immobilized target is then washed with a first solution that removes non-specifically or weakly bound biomolecules. Then the bound antibody polypeptides are eluted with a second solution that includes a saturating amount of free target or a target specific high-affinity competing monoclonal antibody polypeptide, i.e., replicates of the target that are not attached to the particle. The free target binds to biomolecules that dissociate from the target. Rebinding is effectively prevented by the saturating amount of free target relative to the much lower concentration of immobilized target.

The second solution can have solution conditions that are substantially physiological or that are stringent. Typically, the solution conditions of the second solution are identical to the solution conditions of the first solution. Fractions of the second solution are collected in temporal order to distinguish early from late fractions. Later fractions include biomolecules that dissociate at a slower rate from the target than biomolecules in the early fractions.

Further, it is also possible to recover display library members that remain bound to the target even after extended incubation. These can either be dissociated using chaotropic conditions or can be amplified while attached to the target. For example, phage bound to the target can be contacted to bacterial cells.

Selecting or Screening for Specificity.

The display library screening methods described herein can include a selection or screening process that discards display library members that bind to a non-target molecule. Examples of non-target molecules include streptavidin on magnetic beads, blocking agents such as bovine serum albumin, non-fat bovine milk, any capturing or target immobilizing monoclonal antibody polypeptide, or non-transfected cells which do not express the human antigenic target.

In one implementation, a so-called “negative selection” step is used to discriminate between the target and related non-target molecule and related, but distinct non-target molecules. The display library or a pool thereof is contacted to the non-target molecule. Members of the sample that do not bind the non-target are collected and used in subsequent selections for binding to the target molecule or even for subsequent negative selections. The negative selection step can be prior to or after selecting library members that bind to the target molecule.

In another implementation, a screening step is used. After display library members are isolated for binding to the target molecule, each isolated library member is tested for its ability to bind to a non-target molecule (e.g., a non-target listed above). For example, a high-throughput ELISA screen can be used to obtain this data. The ELISA screen can also be used to obtain quantitative data for binding of each library member to the target as well as for cross species reactivity to related targets or subunits of the target and also under different condition such as pH6 or pH 7.5. The non-target and target binding data are compared (e.g., using a computer and software) to identify library members that specifically bind to the target.

Other types of collections of proteins (e.g., expression libraries) can be used to identify proteins with a particular property (e.g., ability to bind an antigen of interest and/or ability to modulate the target), including, e.g., protein arrays of antibody polypeptides (see, e.g., De Wildt et al. (2000) Nat. Biotechnol. 18: 989-994), lambda gt11 libraries, two-hybrid libraries and so forth.

In one embodiment, the library presents a diverse pool of polypeptides, each of which includes an immunoglobulin domain, e.g., an immunoglobulin variable domain. Display libraries are particularly useful, for example, for identifying human or “humanized” antibody polypeptides that recognize human antigens. Such antibody polypeptides can be used as therapeutics to treat human disorders such as autoimmune disorders. Because the constant and framework regions of the antibody polypeptide are human, these therapeutic antibody polypeptides may avoid themselves being recognized and targeted as antigens. The constant regions may also be optimized to recruit effector functions of the human immune system. The in vitro display selection process surmounts the inability of a normal human immune system to generate antibody polypeptides against self-antigens.

A typical antibody display library displays a polypeptide that includes a VH domain and a VL domain. An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay, 1988, Ann. Rev. Immunol. 6:381-405). The display library can display the antibody as a crossover single chain Fv. Other formats can also be used.

As in the case of the xscFvs and other formats, the displayed antibody polypeptide can include one or more constant regions attached to the light and/or heavy chain. In one embodiment, each chain includes one constant region, e.g., as in the case of a Fab. In other embodiments, additional constant regions are displayed.

Antibody polypeptide libraries can be constructed by a number of processes (see, e.g., de Haard et al., 1999, J Biol. Chem. 274:18218-30; Hoogenboom et al., 1998, Immunotechnology 4: 1-20; and Hoogenboom et al., 2000, Immunol. Today 21:371-378. Further, elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. In one embodiment, variation is introduced into all three CDRs of a given variable domain. In another embodiment, the variation is introduced into CDR1 and CDR2, e.g., of a heavy chain variable domain. Any combination is feasible. In one process, antibody polypeptide libraries are constructed by inserting diverse oligonucleotides that encode CDRs into the corresponding regions of the nucleic acid. The oligonucleotides can be synthesized using monomeric nucleotides or trinucleotides. For example, Knappik et al., 2000, J. Mol. Biol. 296:57-86 describe a method for constructing CDR encoding oligonucleotides using trinucleotide synthesis and a template with engineered restriction sites for accepting the oligonucleotides.

In another process, an animal, e.g., a rodent, is immunized with an antigen of interest. The animal is optionally boosted with the antigen to further stimulate the response. Then spleen cells are isolated from the animal, and nucleic acid encoding VH and/or VL domains is amplified and cloned for expression in the display library.

In yet another process, antibody polypeptide libraries are constructed from nucleic acid amplified from naïve germline immunoglobulin genes. The amplified nucleic acid includes nucleic acid encoding the VH and/or VL domain. Sources of immunoglobulin-encoding nucleic acids are described below. Amplification can include PCR, e.g., with primers that anneal to the conserved constant region, or another amplification method.

Nucleic acid encoding immunoglobulin domains can be obtained from the immune cells of, e.g., a human, a primate, mouse, rabbit, camel, llama or rodent. In one example, the cells are selected for a particular property. B cells at various stages of maturity can be selected. In another example, the B cells are naïve.

In one embodiment, fluorescent-activated cell sorting (FACS) is used to sort B cells that express surface-bound IgM, IgD, or IgG molecules. Further, B cells expressing different isotypes of IgG can be isolated. In another embodiment, the B or T cell is cultured in vitro.

The cells can be stimulated in vitro, e.g., by culturing with feeder cells or by adding mitogens or other modulatory reagents, such as antibody polypeptides to CD40, CD40 ligand or CD20, phorbol myristate acetate, bacterial lipopolysaccharide, concanavalin A, phytohemagglutinin, or pokeweed mitogen.

In still one embodiment, the cells are isolated from a subject that has disease or disorder.

In one embodiment, the cells have activated a program of somatic hypermutation. Cells can be stimulated to undergo somatic mutagenesis of immunoglobulin genes, for example, by treatment with anti-immunoglobulin, anti-CD40, and anti-CD38 antibody polypeptides (see, e.g., Bergthorsdottir et al., 2001, J. Immunol. 166:2228). In one embodiment, the cells are naïve.

The nucleic acid encoding an immunoglobulin variable domain can be isolated from a natural repertoire by the following exemplary method. First, RNA is isolated from the immune cell. Full length (i.e., capped) mRNAs are separated (e.g., by degrading uncapped RNAs with calf intestinal phosphatase). The cap is then removed with tobacco acid pyrophosphatase and reverse transcription is used to produce the cDNAs.

The reverse transcription of the first (antisense) strand can be done in any manner with any suitable primer. See, e.g., de Haard et al., 1999, J. Biol. Chem. 274: 18218-30. The primer binding region can be constant among different immunoglobulins, e.g., in order to reverse transcribe different isotypes of immunoglobulin. The primer binding region can also be specific to a particular isotype of immunoglobulin. Typically, the primer is specific for a region that is 3′ to a sequence encoding at least one CDR. In one embodiment, poly-dT primers may be used (and may be preferred for the heavy-chain genes).

A synthetic sequence can be ligated to the 3′ end of the reverse transcribed strand. The synthetic sequence can be used as a primer binding site for binding of the forward primer during PCR amplification after reverse transcription. The use of the synthetic sequence can obviate the need to use a pool of different forward primers to fully capture the available diversity.

The variable domain-encoding gene is then amplified, e.g., using one or more rounds. If multiple rounds are used, nested primers can be used for increased fidelity. The amplified nucleic acid is then cloned into a display library vector.

6. Secondary Screening Methods

After selecting candidate library members that bind to a target, each candidate library member can be further analyzed, e.g., to further characterize its binding properties for the target. Each candidate library member can be subjected to one or more secondary screening assays. The assay can be for a binding property, a catalytic property, an inhibitory property, a physiological property (e.g., cytotoxicity, renal clearance, immunogenicity), a structural property (e.g., stability, conformation, oligomerization state) or another functional property. The same assay can be used repeatedly, but with varying conditions, e.g., to determine pH, ionic, or thermal sensitivities.

As appropriate, the assays can use a display library member directly, a recombinant polypeptide produced from the nucleic acid encoding the selected polypeptide, or a synthetic peptide synthesized based on the sequence of the selected polypeptide. Exemplary assays for binding properties include the following.

ELISA.

Antibody polypeptides selected from an expression library can also be screened for a binding property using an ELISA. For example, each antibody polypeptide is contacted to a microtitre plate whose bottom surface has been coated with the target, e.g., a limiting amount of the target. The plate is washed with buffer to remove non-specifically bound polypeptides. Then the amount of the antibody polypeptide bound to the plate is determined by probing the plate with an antibody polypeptide that can recognize the test antibody polypeptide, e.g., a tag or constant portion of the antibody polypeptide. The detection antibody polypeptide is linked to an enzyme such as alkaline phosphatase or horse radish peroxidase (HRP) which produces a calorimetric product when appropriate substrates are provided.

In the case of an antibody polypeptide from a display library, the antibody polypeptide can be purified from cells or assayed in a display library format, e.g., as a fusion to a filamentous bacteriophage coat. In another version of the ELISA, each antibody polypeptide selected from an expression library is used to coat a different well of a microtitre plate. The ELISA then proceeds using a constant target molecule to query each well.

Homogeneous Binding Assays.

The binding interaction of candidate antibody polypeptide with a target can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means well known in the art (e.g., using a fluorimeter). By titrating the amount of the first or second binding molecule, a binding curve can be generated to estimate the equilibrium binding constant.

Another example of a homogenous assay is ALPHASCREEN™ (Packard Bioscience, Meriden Conn.). ALPHASCREEN™ uses two labeled beads. One bead generates singlet oxygen when excited by a laser. The other bead generates a light signal when singlet oxygen diffuses from the first bead and collides with it. The signal is only generated when the two beads are in proximity. One bead can be attached to the display library member, the other to the target. Signals are measured to determine the extent of binding.

The homogenous assays can be performed while the candidate polypeptide is attached to the display library vehicle, e.g., a bacteriophage.

Surface Plasmon Resonance (SPR).

The binding interaction of a molecule isolated from an expression library and a target can be analyzed using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether, 1988, Surface Plasmons Springer Verlag; Sjolander and Urbaniczky, 1991, Anal. Chem. 63:2338-2345; Szabo et al., 1995, Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{d}), and kinetic parameters, including Kₒₙ and K_{off}, for the binding of a biomolecule to a target. Such data can be used to compare different biomolecules. For example, selected proteins from an expression library can be compared to identify proteins that have high affinity for the target or that have a slow K_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow K_{off}. This information can be combined with structural modeling (e.g., using homology modeling, energy minimization, or structure determination by x-ray crystallography or NMR). As a result, an understanding of the physical interaction between the protein and its target can be formulated and used to guide other design processes.

Cellular Assays.

A library of candidate antibody polypeptides (e.g., previously identified by a display library or otherwise) can be screened for target binding on cells which transiently or stably express and display the target of interest on the cell surface. For example, the target can include vector nucleic acid sequences that include segments that encode only the extracellular portion of the polypeptides such that the chimeric target polypeptides are produced within the cell, secreted from the cell, or attached to the cell surface through the anchor e.g., in fusion with a membrane anchoring proteins such as Fc. The cell surface expressed target can be used for screening antibody polypeptides that bind to an antigen of interest and block the binding of IgG-Fc. For example, non-specific human IgG-Fc could be fluorescently labeled and its binding to the antigen in the presence of absence of antagonistic antibody polypeptide can be detected by a change in fluorescence intensity using flow cytometry e.g., a FACS machine.

7. Other Methods for Obtaining Binding Antibody Polypeptides

In addition to the use of display libraries, other methods can be used to obtain an antibody polypeptide that binds an antigen of interest.

In one embodiment, the non-human animal includes at least a part of a human immunoglobulin gene. For example, it is possible to engineer mouse strains deficient in mouse antibody polypeptide production with large fragments of the human Ig loci. Using the hybridoma technology, antigen-specific monoclonal antibody polypeptides (Mabs) derived from the genes with the desired specificity may be produced and selected. See, e.g., XENOMOUSE™, Green et al., 1994, Nat. Gen. 7:13-21; U.S. 2003-0070185, WO 96/34096, published Oct. 31, 1996, and PCT Application No. PCT/US96/05928, filed Apr. 29, 1996.

In one embodiment, a monoclonal antibody polypeptide is obtained from the non-human animal, and then modified, e.g., humanized or deimmunized. Winter describes a CDR-grafting method that may be used to prepare the humanized antibody polypeptides (UK Patent Application GB 2188638A, filed on Mar. 26, 1987; U.S. Pat. No. 5,225,539. All of the CDRs of a particular human antibody polypeptide may be replaced with at least a portion of a non-human CDR or alternatively some of the CDRs may be replaced with non-human CDRs. In some embodiments, it is only necessary to replace the number of CDRs required for binding of the humanized antibody polypeptide to a predetermined antigen.

Humanized antibody polypeptides can be generated by replacing sequences of the Fv variable region that are not directly involved in antigen binding with equivalent sequences from human Fv variable regions. General methods for generating humanized antibody polypeptides are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody polypeptide against a predetermined target, as described above. The recombinant DNA encoding the humanized antibody polypeptide, or fragment thereof, can then be cloned into an appropriate expression vector.

An antibody polypeptide may also be modified by specific deletion of human T cell epitopes or “deimmunization” by the methods disclosed in WO 98/52976 and WO 00/34317, the contents of which are specifically incorporated by reference herein. Briefly, the heavy and light chain variable regions of an antibody polypeptide can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable regions or by single amino acid substitutions. As far as possible conservative substitutions are made, often but not exclusively, an amino acid common at this position in human germline antibody polypeptide sequences may be used. Human germline sequences are disclosed in Tomlinson, I. A. et al., 1992, J. Mol. Biol. 227:776-798; Cook, G. P. et al., 1995, Immunol. Today Vol. 16 (5): 237-242; Chothia, D. et al., 1992, 1 Mol. Bio. 227:799-817. The V BASE directory provides a comprehensive directory of human immunoglobulin variable region sequences (compiled by Tomlinson, I. A. et al. MRC Centre for Protein Engineering, Cambridge, UK). After the deimmunizing changes are identified, nucleic acids encoding V_{H} and V_{L} can be constructed by mutagenesis or other synthetic methods (e.g., de novo synthesis, cassette replacement, and so forth). Mutagenized variable sequence can, optionally, be fused to a human constant region, e.g., human IgG1 or κ constant regions.

In some cases, a potential T cell epitope will include residues which are known or predicted to be important for antibody polypeptide function. For example, potential T cell epitopes are usually biased towards the CDRs. In addition, potential T cell epitopes can occur in framework residues important for antibody polypeptide structure and binding. Changes to eliminate these potential epitopes will in some cases require more scrutiny, e.g., by making and testing chains with and without the change. Where possible, potential T cell epitopes that overlap the CDRs were eliminated by substitutions outside the CDRs. In some cases, an alteration within a CDR is the only option, and thus variants with and without this substitution should be tested. In other cases, the substitution required to remove a potential T cell epitope is at a residue position within the framework that might be beneficial for antibody polypeptide binding. In these cases, variants with and without this substitution should be tested. Thus, in some cases several variant deimmunized heavy and light chain variable regions were designed and various heavy/light chain combinations tested in order to identify the optimal deimmunized antibody polypeptide. The choice of the final deimmunized antibody polypeptide can then be made by considering the binding affinity of the different variants in conjunction with the extent of deimmunization, i.e., the number of potential T cell epitopes remaining in the variable region. Deimmunization can be used to modify any antibody polypeptide, e.g., an antibody that includes a non-human sequence, e.g., a synthetic antibody, a murine antibody other non-human monoclonal antibody, or an antibody isolated from a display library.

8. Pharmaceutical Compositions and Assay Systems for Candidate Antibody Polypeptides

A pharmaceutical composition comprises a therapeutically-effective amount of one or more antibody polypeptides and optionally a pharmaceutically acceptable carrier. Pharmaceutically acceptable carriers include, for example, water, saline, phosphate buffered saline, dextrose, glycerol, ethanol and the like, as well as combinations thereof. Pharmaceutically acceptable carriers can further comprise minor amounts of auxiliary substances, such as wetting or emulsifying agents, preservatives, or buffers that enhance the shelf-life or effectiveness of the fusion protein. The compositions can be formulated to provide quick, sustained, or delayed release of the active ingredient(s) after administration. Suitable pharmaceutical compositions and processes for preparing them are well known in the art. See, e.g., Remington, THE SCIENCE AND PRACTICE OF PHARMACY, A. Gennaro, et al., eds., 21^{st} ed., Mack Publishing Co. (2005).

A method of treating a disease in a patient in need of such treatment may comprise administering to the patient a therapeutically effective amount of the pharmaceutical composition. As used herein, a “patient” means an animal, e.g. mammal, including humans. “Treatment” or “treat” or “treating” refers to the process involving alleviating the progression or severity of a symptom, disorder, condition, or disease.

The pharmaceutical composition may be administered alone or in combination therapy, (i.e., simultaneously or sequentially) with an another pharmaceutical agent. Different diseases can require use of specific auxiliary compounds useful for treating diseases, which can be determined on a patient-to-patient basis. For example, the pharmaceutical composition may be administered in combination with one or more suitable adjuvants known in the art.

Any suitable method or route can be used to administer the antibody polypeptide or the pharmaceutical composition. Routes of administration include, for example, oral, intravenous, intraperitoneal, subcutaneous, or intramuscular administration. A therapeutically effective dose of administered antibody polypeptide(s) depends on numerous factors, including, for example, the type and severity of the disease being treated, the use of combination therapy, the route of administration of the antibody polypeptide(s) or pharmaceutical composition, and the weight of the patient.

Candidate antibody polypeptides that bind an antigen of interest can be further characterized in assays that measure their modulatory activity toward the target or fragments thereof in vitro or in vivo. For example, the antigen can be combined with a substrate such as non-specific IgG or Fc portion of the IgG or albumin under assay conditions permitting reaction of the antigen with the substrate. The assay is performed in the absence of the antigen candidate antibody polypeptide, and in the presence of increasing concentrations of the antigen candidate antibody polypeptide. The concentration of candidate antibody polypeptide at which 50% of the target's activity (e.g., binding to the substrate) is inhibited by the candidate antibody polypeptide is the IC₅₀ value (Inhibitory Concentration 50%) or EC₅₀ (Effective Concentration 50%) value for that antibody. Within a series or group of candidate antibody polypeptides, those having lower IC₅₀ or EC₅₀ values are considered more potent inhibitors of the antigen than those antibody polypeptides having higher IC₅₀ or EC₅₀ values. In some embodiments, antibody polypeptides have an IC₅₀ value of 800 nM, 400 nM, 100 nM, 25 nM, 5 nM, 1 nM, or less as measured in an in vitro assay for inhibition of target activity.

The candidate antibody polypeptides can also be evaluated for selectivity toward the antigen. For example, a candidate antibody polypeptide can be assayed for its potency toward the antigen and a panel of cell surface receptors, such as receptors that also utilize the 132M domain, and an IC₅₀ value or EC₅₀ value can be determined for each receptor protein. In one embodiment, a compound that demonstrates a low IC₅₀ value or EC₅₀ value for the target, and a higher IC₅₀ value or EC₅₀ value for other receptors within the test panel (e.g., MHC class I molecules) is considered to be selective toward the antigen.

Ex vivo endothelial cells or epithelial cells expressing the endogenous the antigen could be used to follow the endocytosis or transcytosis of the candidate antibody polypeptides under different pH and temperature conditions. IgG transcytosis or recycling by the antigen can be measured by following a labeled antibody polypeptide in the presence or absence of various chemicals and under different conditions that are known to influence or affect the intracellular trafficking pathway.

A pharmacokinetics study in rat, mice, or monkey could be performed with pH dependent and independent antigen binding antibody polypeptides for determining their half-life in the serum. Likewise, the protective effect of the antibody polypeptide can be assessed in vivo for potential use in immunomodulating therapy or as an salvage immunotherapy by injecting the antibody polypeptide in the presence or absence of a labeled IgG or the labeled Fc portion of the IgG. A decrease in the half-life of the labeled IgG/Fc in the presence of the candidate antibody polypeptide is an indication of the therapeutic efficacy of the antibody.

### EXAMPLES

### Example 1

### Trastuzumab xscFv Design

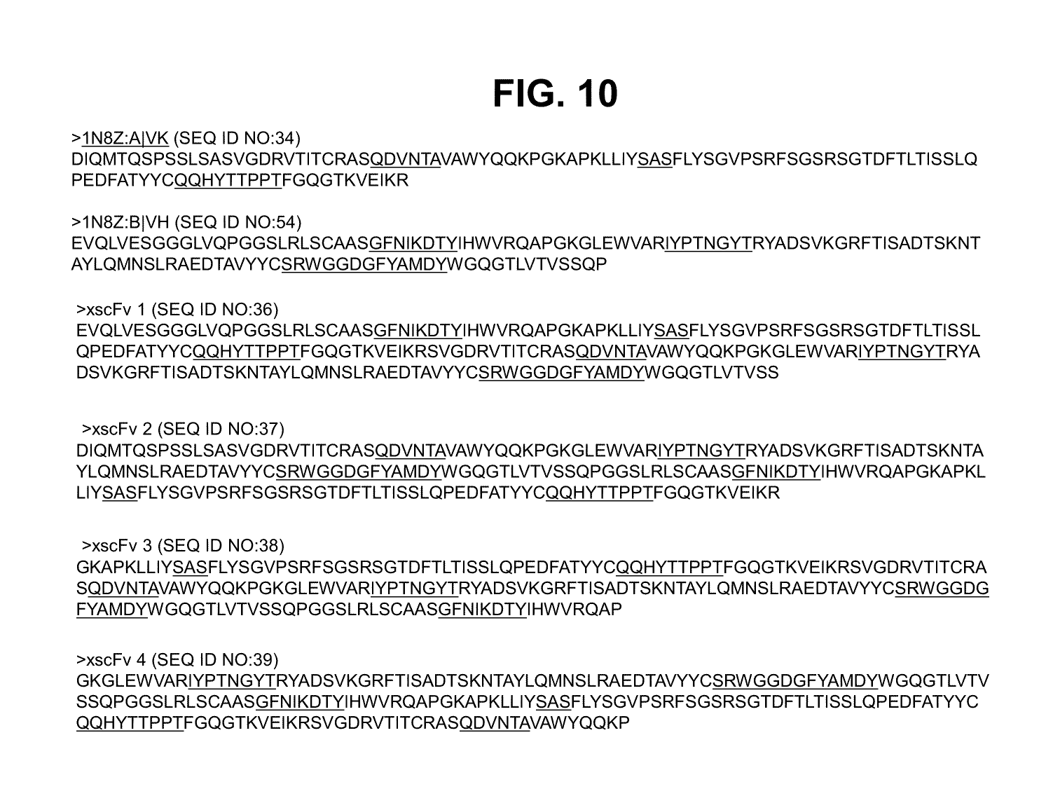

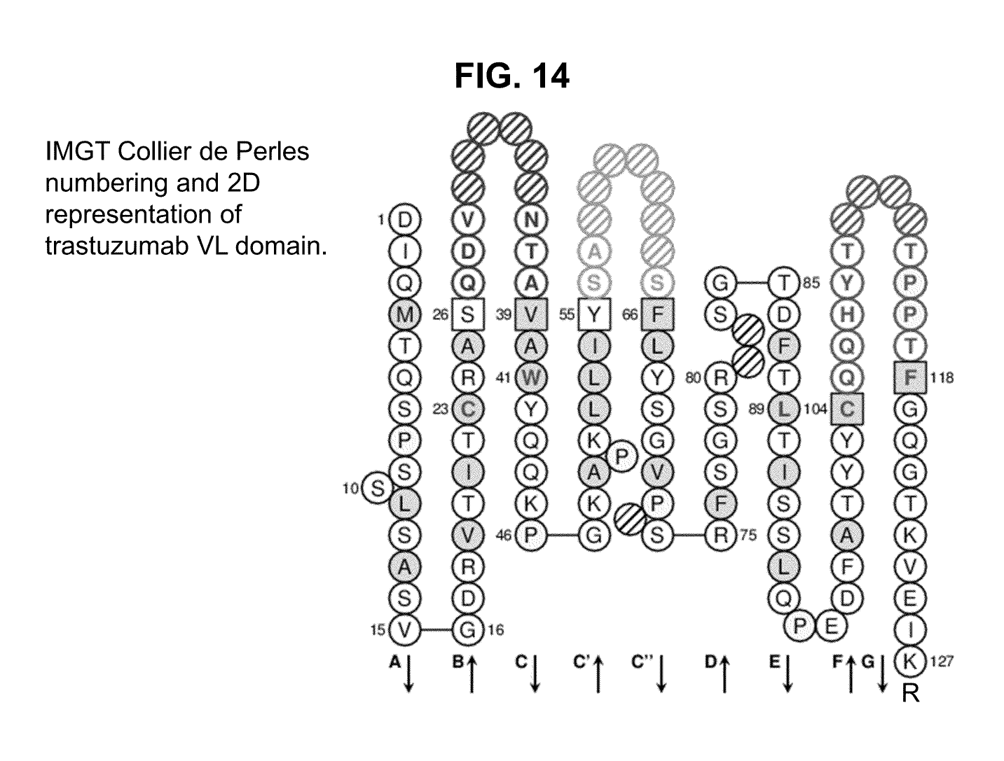

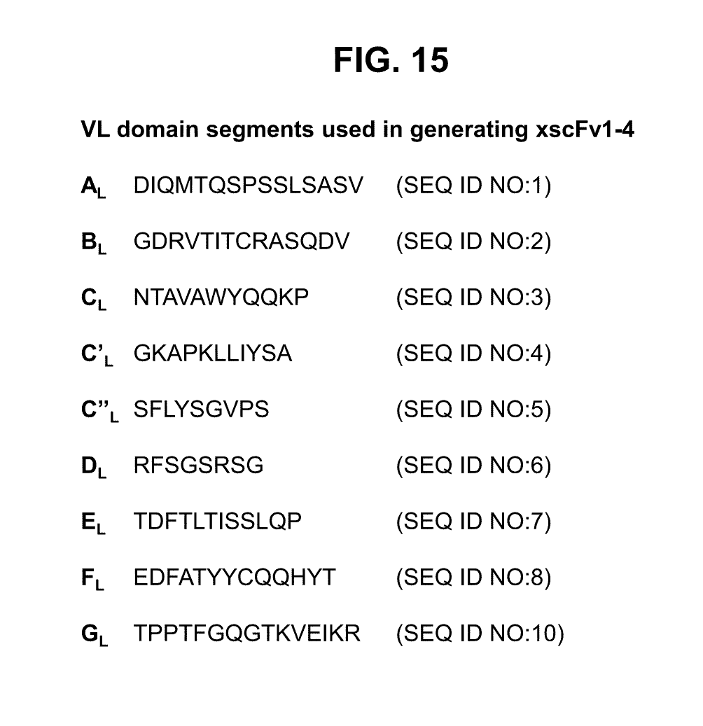

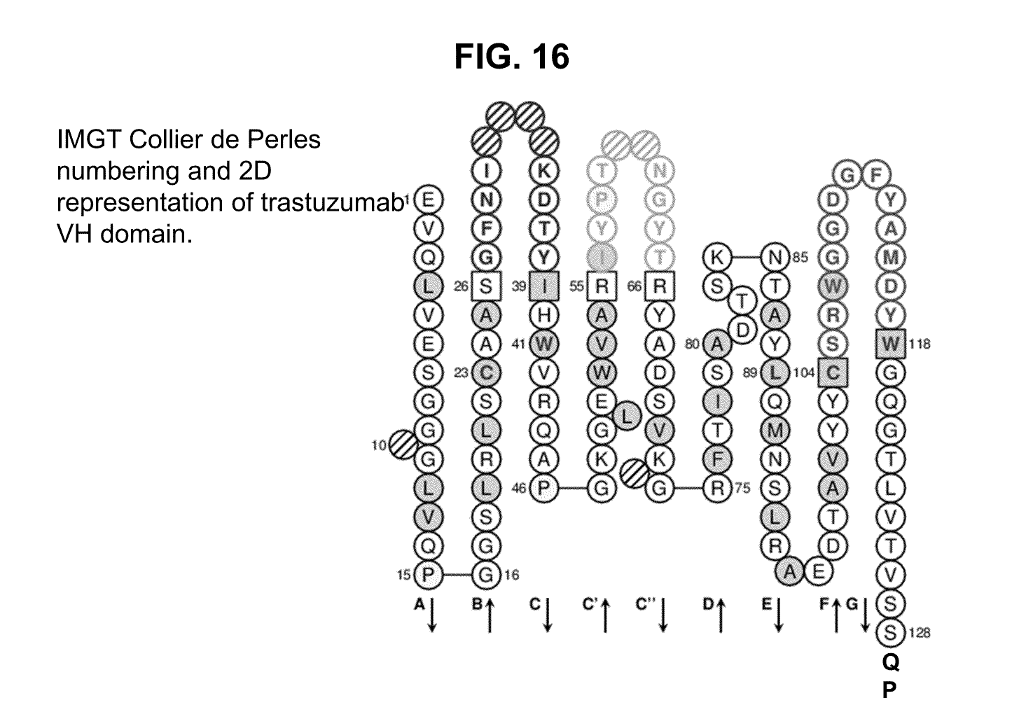

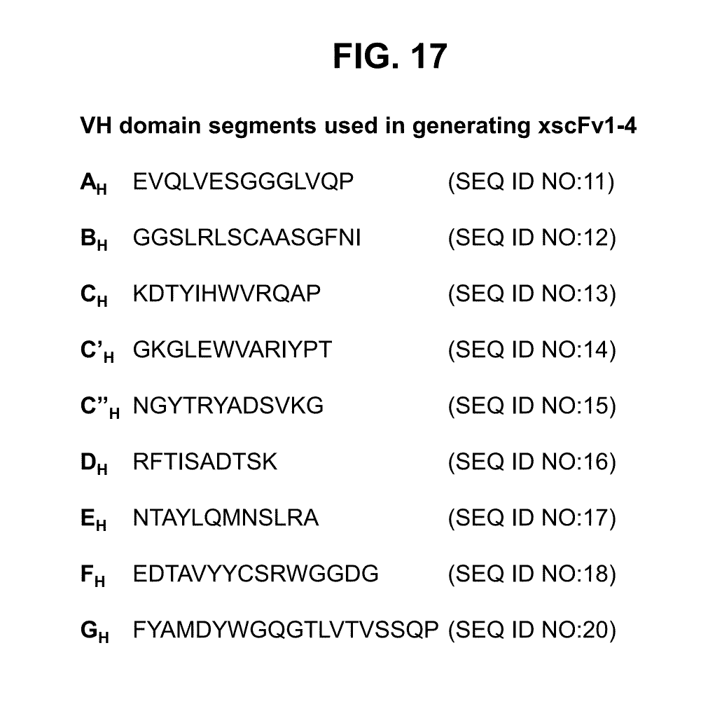

Potential trastuzumab interdomain and intradomain crossover points were assessed based on manual observation of trastuzumab scFv structure and general scFv structure. Crossover points were selected based on: 1) regions of flexibility (i.e., loops); 2) in close proximity (9-12 Å); 3) with compatible sequence directionality (N→C flow of sequence); 4) in a region where a crossover event would allow for maximal continuation of the chain; and 5) at points that are minimally disruptive to the CDR sequence. Loop C-C′ on the heavy chain (QAPGKG; SEQ ID NO:28) and loop C-C′ on the light chain (QPKGKA; SEQ ID NO:29) were selected as regions for interdomain crossovers. The N terminus to C terminus directionality of the loops was conserved. The loops between strands G and B with G→B directionality were used for light chain intradomain crossovers. The sequence after light chain strand G can be defined as (VEIKRTV; SEQ ID NO:30) and the sequence preceding light chain strand B can be defined as (ASVGDR; SEQ ID NO:31). The loops between strands G and B with G→B directionality were used for heavy chain intradomain crossovers. The sequence after heavy chain strand G can be defined as (TVSSASTK; SEQ ID NO:32) and the sequence preceding heavy chain strand B can be defined as (VQPGGS; SEQ ID NO:33). For heavy chain intradomain crossovers, therefore, “QP” was added to the C-terminal end of strand G. See G_{H} strand in FIG. 17. “QP” is also shown added to the C-terminal of V_{H} in FIG. 10. Four xscFv trastuzumab were constructed. The native Trastuzumab heavy chain and light chain amino acid sequences, and the final, crossed-over xscFv amino acid sequences are listed in Table 1 below (CDRs are underlined) and in FIG. 10. See also FIGS. 14-17.

TABLE 1
Trastuzumab xscFv Amino Acid Sequences

| Model | Amino Acid Sequence |
|---|---|
| Trastuzumab | DIQMTQSPSSLSASVGDRVTITCRASQDVNTAVAWYQQKPGK |
| V_{L} | APKLLIYSASFLYSGVPSRFSGSRSGTDFTLTISSLQPEDFATYYC |
| (1N8Z:A\|VK) | QQHYTTPPTFGQGTKVEIKR |
| | (SEQ ID NO: 34) |
| Trastuzumab | EVQLVESGGGLVQPGGSLRLSCAASGFNIKDTYIHWVRQAPGK |
| V_{H} | GLEWVARIYPTNGYTRYADSVKGRFTISADTSKNTAYLQMNSL |
| (1N8Z:B\|VH) | RAEDTAVYYCSRWGGDGFYAMDYWGQGTLVTVSS |
| | (SEQ ID NO: 35) |
| xscFv 1 | EVQLVESGGGLVQPGGSLRLSCAASGFNIKDTYIHWVRQAPGK |
| | APKLLIYSASFLYSGVPSRFSGSRSGTDFTLTISSLQPEDFATYY |
| | CQQHYTTPPTFGQGTKVEIKRSVGDRVTITCRASQDVNTAVA |
| | WYQQKPGKGLEWVARIYPTNGYTRYADSVKGRFTISADTSKN |
| | TAYLQMNSLRAEDTAVYYCSRWGGDGFYAMDYWGQGTLVTVSS |
| | (SEQ ID NO: 36) |
| xscFv 2 | DIQMTQSPSSLSASVGDRVTITCRASQDVNTAVAWYQQKPGKGL |
| | EWVARIYPTNGYTRYADSVKGRFTISADTSKNTAYLQMNSLRAED |
| | TAVYYCSRWGGDGFYAMDYWGQGTLVTVSSQPGGSLRLSCAAS |
| | GFNIKDTYIHWVRQAPGKAPKLLIYSASFLYSGVPSRFSGSRSGTD |
| | FTLTISSLQPEDFATYYCQQHYTTPPTFGQGTKVEIKR |
| | (SEQ ID NO: 37) |
| xscFv 3 | GKAPKLLIYSASFLYSGVPSRFSGSRSGTDFTLTISSLQPEDFATYYC |
| | QQHYTTPPTFGQGTKVEIKRSVGDRVTITCRASQDVNTAVAWYQQ |
| | KPGKGLEWVARIYPTNGYTRYADSVKGRFTISADTSKNTAYLQMN |
| | SLRAEDTAVYYCSRWGGDGFYAMDYWGQGTLVTVSSQPGGSLRL |
| | SCAASGFNIKDTYIHWVRQAP |
| | (SEQ ID NO: 38) |
| xscFv 4 | GKGLEWVARIYPTNGYTRYADSVKGRFTISADTSKNTAYLQMNSLR |
| | AEDTAVYYCSRWGGDGFYAMDYWGQGTLVTVSSQPGGSLRLSCAA |
| | SGFNIKDTYIHWVRQAPGKAPKLLIYSASFLYSGVPSRFSGSRSGTD |
| | FTLTISSLQPEDFATYYCQQHYTTPPTFGQGTKVEIKRSVGDRVT |
| | ITCRASQDVNTAVAWYQQKP |
| | (SEQ ID NO: 39) |

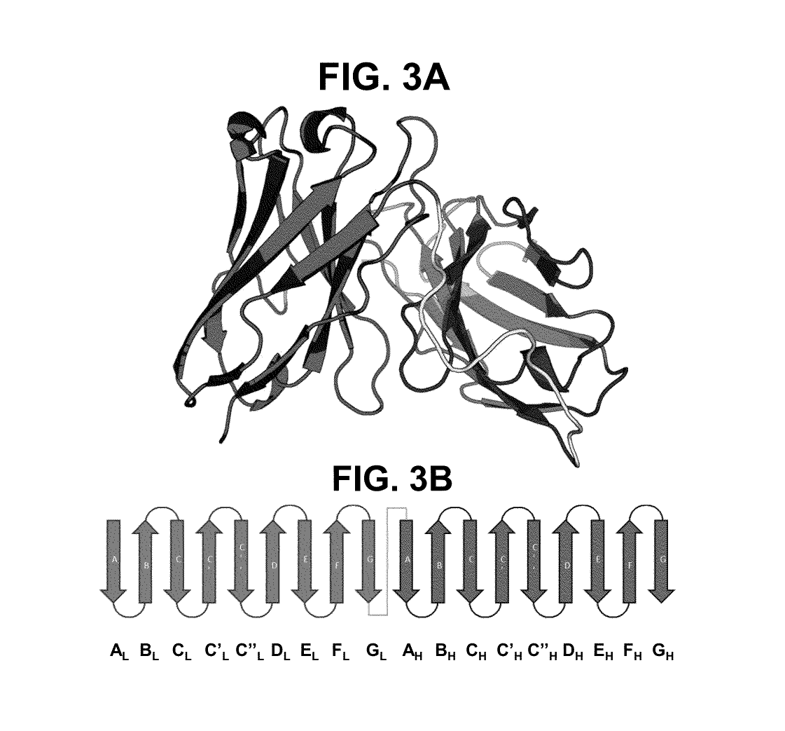

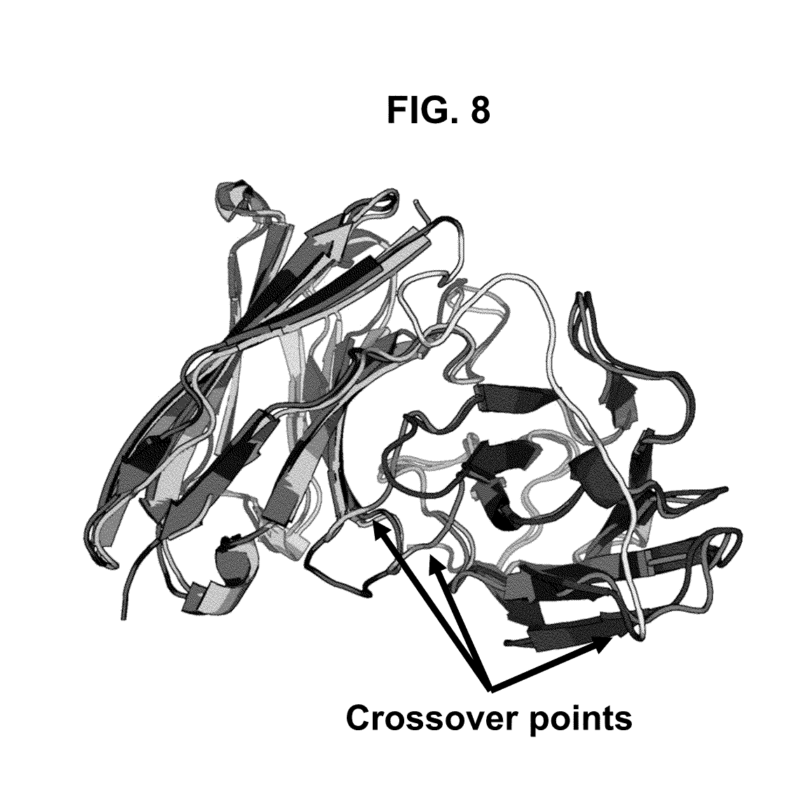

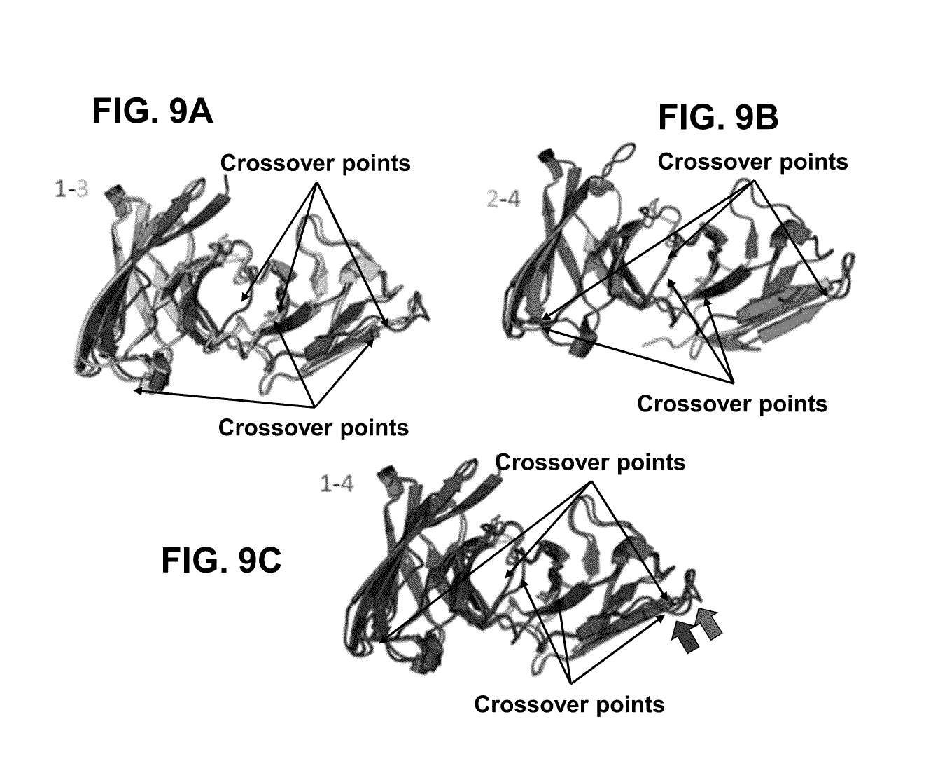

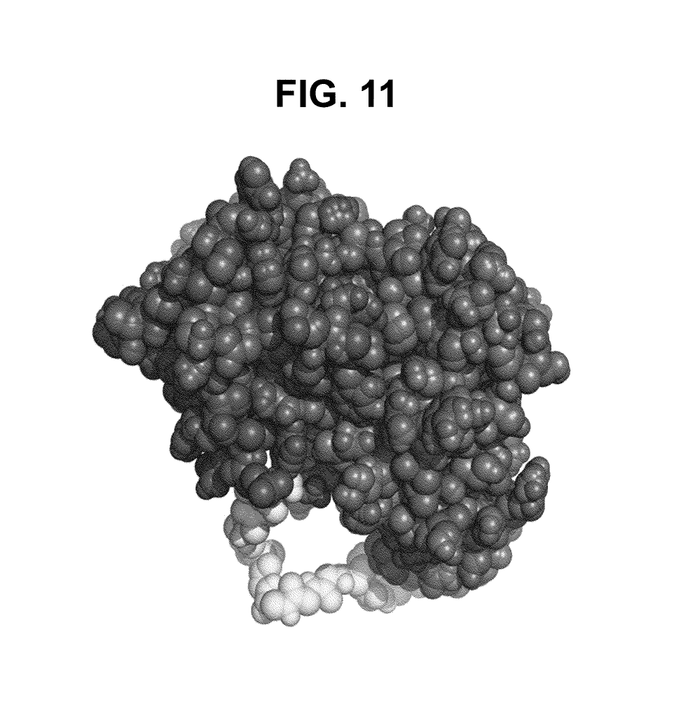

The three dimensional and two dimensional predicted structures of trastuzumab xscFvs 1-4 (as compared to standard scFvs) are depicted schematically in FIGS. 1-9 and 11.

### Example 2

### Vector Design and Mammalian Transfection

ScFv-Fc fragments encoding Trastuzumab and xscFv-Fc Trastuzumab were cloned into constitutive expression vectors. Three to five μg of vector DNA was digested using AleI, ClaI and NheI restriction enzymes (New England BioLabs) per manufacturer's recommendations. Double digests were performed. Digests were run on a 1% agarose gel run in TAE buffer until good band separation was achieved. The desired band was then excised from the gel and purified using the Qiagen Gel Extraction Kit run on a QiaCube (Qiagen). DNA was resuspended in Qiagen Elution Buffer (EB). DNA concentrations were quantified on a Nanodrop.

In-Fusion (Clontech) cloning reactions were performed as per manufacturer's recommendations. 20 ng of insert scFv or xscFv DNA was mixed with 20 ng of vector DNA and 2 μL of In-Fusion mix and brought to a total volume of 10 μL with water. The reaction was incubated at 50° C. for 20 minutes. 40 μL of Stellar Competent Cells (Clontech) were mixed with of the In-Fusion reaction and incubated on ice for 30 minutes. The mix was heat shocked at 42° C. for 45 seconds and incubated on ice for 2 minutes. 250 μL of Super Optimal broth with Catabolite repression (SOC) media was added to the mix and incubated at 37° C. for one hour. Cells were concentrated by centrifugation and plated on LB media with appropriate antibiotics.

Generation of VVN-275231

VVN-253964 was digested with NheI and ClaI to remove the open reading frame from a plasmid designed for high level constitutive expression. VVN-185517, which encodes a Trastuzumab (VL-VH) scFv Fc fusion, was digested with NheI and ClaI to extract the open reading frame. The Trastuzumab (VL-VH) scFv Fc fusion open reading frame was ligated into VVN-253964 to create GEU-92, expressing a FLAG-tagged Trastuzumab (VL-VH) scFv Fc fusion. GEU-92 was used as a PCR template with primers ESC-F1 and ESC-R1 (sequences provided in Table 2 below) to generate a PCR product encoding an Fc fragment lacking the FLAG tag:

TABLE 2
Primer Sequences

| Primer | | |
|---|---|---|
| Name | Primer Sequence | Primer Use |
| ESC-F1 | GGTGTCTCCATCTTCCATCGATTTATTTAC | Amplification |
| | (SEQ ID NO: 40) | of Fc region |
| ESC-R1 | TCACCGTCTCCTCGGGATCCGAGCCCAAAT | Amplification |
| | CTTGTGACAAAACTCACACA | of Fc region |
| | (SEQ ID NO: 41) | |

This PCR product was cloned into GEU-92 that had been digested with BamHI and ClaI to remove the tagged Fe region using the In-Fusion cloning kit to create VVN-275231. Positive clones were identified by restriction mapping and the open reading frame was verified by DNA sequencing.

Generation of xscFvs 1-4

VVN-275231 was digested with NheI and AleI to remove the scFv domain of the scFv-Fc fusion. Four inserts were de novo synthesized as a G-Block by IDT. Each synthetic fragment insert (Table 4) was joined with the digested VVN-275231 using In-Fusion. Fragment HO1Fc (SEQ ID NO:46) was used to create VVN-4588153 (xscFv 1-Fe), fragment HO2Fc (SEQ ID NO:47) was used to create VVN-4588154 (xscFv 2-Fe), fragment HO3Fc (SEQ ID NO:48) was used to create VVN-4588159 (xscFv 3-Fe), and fragment HO4Fc (SEQ ID NO:49) was used to create VVN-4588160 (xscFv 4-Fc). Clones were screened by colony PCR using primers ESCR7 and TC1R (Table 3).

TABLE 3
Primers

| Primer | | |
|---|---|---|
| Name | Primer Sequence | Primer Use |
| ESCR7 | AAGGAATTGAGAGCCGCTAGC | Colony PCR |
| | (SEQ ID NO: 42) | |
| TC1R | CGAGGAGACGGTGACCAGGGTTC | Colony PCR |
| | (SEQ ID NO: 43) | |
| 6837F1 | TTTTTGCTAATCCCTTTTGTGTGCTGA | Sequence |
| | (SEQ ID NO: 44) | verification |
| OriSeqR | TGACCACACGGTACGTGCTGTTG | Sequence |
| | (SEQ ID NO: 45) | verification |

Each candidate was sent for complete DNA sequencing of the open reading frame to verify each construct sequence. The construct sequences are provided in Table 4 below.

TABLE 4
Synthetic sequences (G-Blocks, IDT) encoding xscFv fragments

| Fragment name | Fragment sequence |
|---|---|
| HO1Fc | GAAAAAAGGAATTGAGAGCCGCTAGCGCCACCATGAGGCTCCCTGCT |
| | CAGCTCCTGGGGCTGCTAATGCTCTGGGTCCCAGGCTCCAGTGGGGA |
| | GGTTCAGCTGGTGGAGTCTGGCGGTGGCCTGGTGCAGCCAGGGGGCT |
| | CACTCCGTTTGTCCTGTGCAGCTTCTGGCTTCAACATTAAAGACACCT |
| | ATATACACTGGGTGCGTCAGGCCCCGGGAAAAGCTCCGAAACTACTG |
| | ATTTACTCGGCATCCTTCCTCTACTCTGGAGTCCCTTCTCGCTTCTCTG |
| | GTTCCAGATCTGGGACGGATTTCACTCTGACCATCAGCAGTCTGCAG |
| | CCGGAAGACTTCGCAACTTATTACTGTCAGCAACATTATACTACTCCT |
| | CCCACGTTCGGACAGGGTACCAAGGTGGAGATCAAACGTTCTGTGGG |
| | CGATAGGGTCACCATCACCTGCCGTGCCAGTCAGGATGTGAATACTG |
| | CTGTAGCCTGGTATCAACAGAAACCAGGTAAGGGCCTGGAATGGGTT |
| | GCAAGGATTTATCCTACGAATGGTTATACTAGATATGCCGATAGCGT |
| | CAAGGGCCGTTTCACTATAAGCGCAGACACATCCAAAAACACAGCCT |
| | ACCTGCAGATGAACAGCCTGCGTGCTGAGGACACTGCCGTCTATTAT |
| | TGTTCTAGATGGGGAGGGGACGGCTTCTATGCTATGGACTACTGGGG |
| | TCAAGGAACCCTGGTCACCGTCTCCTCGGGATCCGAGCCCAAATCTA |
| | GCGACAAAACTCACACATGCCCACCGTGCCCAGCACCTGAACTCCTG |
| | GGGGGACCGTCAGTCTTCCTCTTCCCCCCAAAACCCAAGGACACCCT |
| | CATGATCTCCCGGACCCCTGAGGTCACATGCGTGGTGGTGGACGTG |
| | (SEQ ID NO: 46) |
| HO2Fc | GAAAAAAGGAATTGAGAGCCGCTAGCGCCACCATGAGGCTCCCTGCT |
| | CAGCTCCTGGGGCTGCTAATGCTCTGGGTCCCAGGCTCCAGTGGGGA |
| | TATCCAGATGACCCAGTCCCCGAGCTCCCTGTCCGCCTCTGTGGGCG |
| | ATAGGGTCACCATCACCTGCCGTGCCAGTCAGGATGTGAATACTGCT |
| | GTAGCCTGGTATCAACAGAAACCAGGTAAGGGCCTGGAATGGGTTGC |
| | AAGGATTTATCCTACGAATGGTTATACTAGATATGCCGATAGCGTCA |
| | AGGGCCGTTTCACTATAAGCGCAGACACATCCAAAAACACAGCCTAC |
| | CTGCAGATGAACAGCCTGCGTGCTGAGGACACTGCCGTCTATTATTG |
| | TTCTAGATGGGGAGGGGACGGCTTCTATGCTATGGACTACTGGGGTC |
| | AAGGAACCCTGGTCACCGTCTCCTCGCAGCCAGGGGGCTCACTCCGT |
| | TTGTCCTGTGCAGCTTCTGGCTTCAACATTAAAGACACCTATATACAC |
| | TGGGTGCGTCAGGCCCCGGGAAAAGCTCCGAAACTACTGATTTACTC |
| | GGCATCCTTCCTCTACTCTGGAGTCCCTTCTCGCTTCTCTGGTTCCAG |
| | ATCTGGGACGGATTTCACTCTGACCATCAGCAGTCTGCAGCCGGAAG |
| | ACTTCGCAACTTATTACTGTCAGCAACATTATACTACTCCTCCCACGT |
| | TCGGACAGGGTACCAAGGTGGAGATCAAACGTGGATCCGAGCCCAA |
| | ATCTAGCGACAAAACTCACACATGCCCACCGTGCCCAGCACCTGAAC |
| | TCCTGGGGGGACCGTCAGTCTTCCTCTTCCCCCCAAAACCCAAGGAC |
| | ACCCTCATGATCTCCCGGACCCCTGAGGTCACATGCGTGGTGGTGGA |
| | CGTG |
| | (SEQ ID NO: 47) |
| HO3Fc | GAAAAAAGGAATTGAGAGCCGCTAGCGCCACCATGAGGCTCCCTGCT |
| | CAGCTCCTGGGGCTGCTAATGCTCTGGGTCCCAGGCTCCAGTGGGGG |
| | AAAAGCTCCGAAACTACTGATTTACTCGGCATCCTTCCTCTACTCTGG |
| | AGTCCCTTCTCGCTTCTCTGGTTCCAGATCTGGGACGGATTTCACTCT |
| | GACCATCAGCAGTCTGCAGCCGGAAGACTTCGCAACTTATTACTGTC |
| | AGCAACATTATACTACTCCTCCCACGTTCGGACAGGGTACCAAGGTG |
| | GAGATCAAACGTTCTGTGGGCGATAGGGTCACCATCACCTGCCGTGC |
| | CAGTCAGGATGTGAATACTGCTGTAGCCTGGTATCAACAGAAACCAG |
| | GTAAGGGCCTGGAATGGGTTGCAAGGATTTATCCTACGAATGGTTAT |
| | ACTAGATATGCCGATAGCGTCAAGGGCCGTTTCACTATAAGCGCAGA |
| | CACATCCAAAAACACAGCCTACCTGCAGATGAACAGCCTGCGTGCTG |
| | AGGACACTGCCGTCTATTATTGTTCTAGATGGGGAGGGGACGGCTTC |
| | TATGCTATGGACTACTGGGGTCAAGGAACCCTGGTCACCGTCTCCTC |
| | GCAGCCAGGGGGCTCACTCCGTTTGTCCTGTGCAGCTTCTGGCTTCAA |
| | CATTAAAGACACCTATATACACTGGGTGCGTCAGGCCCCGGGATCCG |
| | AGCCCAAATCTAGCGACAAAACTCACACATGCCCACCGTGCCCAGCA |
| | CCTGAACTCCTGGGGGGACCGTCAGTCTTCCTCTTCCCCCCAAAACCC |
| | AAGGACACCCTCATGATCTCCCGGACCCCTGAGGTCACATGCGTGGT |
| | GGTGGACGTG |
| | (SEQ ID NO: 48) |
| HO4Fc | GAAAAAAGGAATTGAGAGCCGCTAGCGCCACCATGAGGCTCCCTGCT |
| | CAGCTCCTGGGGCTGCTAATGCTCTGGGTCCCAGGCTCCAGTGGGGG |
| | TAAGGGCCTGGAATGGGTTGCAAGGATTTATCCTACGAATGGTTATA |
| | CTAGATATGCCGATAGCGTCAAGGGCCGTTTCACTATAAGCGCAGAC |
| | ACATCCAAAAACACAGCCTACCTGCAGATGAACAGCCTGCGTGCTGA |
| | GGACACTGCCGTCTATTATTGTTCTAGATGGGGAGGGGACGGCTTCT |
| | ATGCTATGGACTACTGGGGTCAAGGAACCCTGGTCACCGTCTCCTCG |
| | CAGCCAGGGGGCTCACTCCGTTTGTCCTGTGCAGCTTCTGGCTTCAAC |
| | ATTAAAGACACCTATATACACTGGGTGCGTCAGGCCCCGGGAAAAGC |
| | TCCGAAACTACTGATTTACTCGGCATCCTTCCTCTACTCTGGAGTCCC |
| | TTCTCGCTTCTCTGGTTCCAGATCTGGGACGGATTTCACTCTGACCAT |
| | CAGCAGTCTGCAGCCGGAAGACTTCGCAACTTATTACTGTCAGCAAC |
| | ATTATACTACTCCTCCCACGTTCGGACAGGGTACCAAGGTGGAGATC |
| | AAACGTTCTGTGGGCGATAGGGTCACCATCACCTGCCGTGCCAGTCA |
| | GGATGTGAATACTGCTGTAGCCTGGTATCAACAGAAACCAGGATCCG |
| | AGCCCAAATCTAGCGACAAAACTCACACATGCCCACCGTGCCCAGCA |
| | CCTGAACTCCTGGGGGGACCGTCAGTCTTCCTCTTCCCCCCAAAACCC |
| | AAGGACACCCTCATGATCTCCCGGACCCCTGAGGTCACATGCGTGGT |
| | GGTGGACGTG |
| | (SEQ ID NO: 49) |

One day before transfection, 293T cells were plated into T-150 flask containing 40 ml of growth medium without antibiotics. Cells were cultured until they were 70-90% confluent at the time of transfection. Antibody xscFv-Fc plasmid DNA (˜60 μg) and ˜150 μl Lipofectamine 2000® was diluted in 3.0 ml Opti-MEM® I Reduced Serum Medium. Diluted plasmid DNA was combined with diluted Lipofectamine® 2000 and incubated for 20 minutes at room temperature. 6 ml of the DNA/Lipofectamine complex was added to each T150 flask and mix gently. Cells were incubated at 37° C. in a CO₂ incubator for 48 hours, prior to testing for trastuzumab xscFv expression and purification.

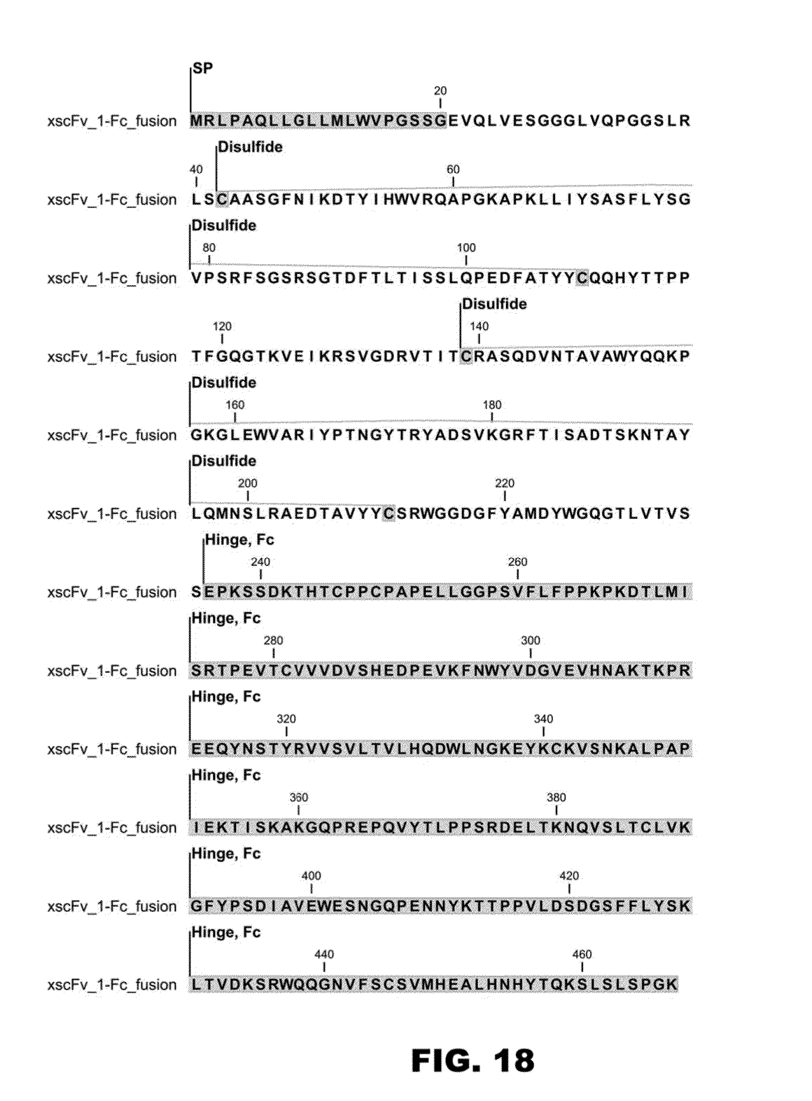

TABLE 5
Examples of Polypeptide Sequences of Four xscFv-Fc Fusion Proteins
CDRs are underlined. See also, FIGS. 18-21.

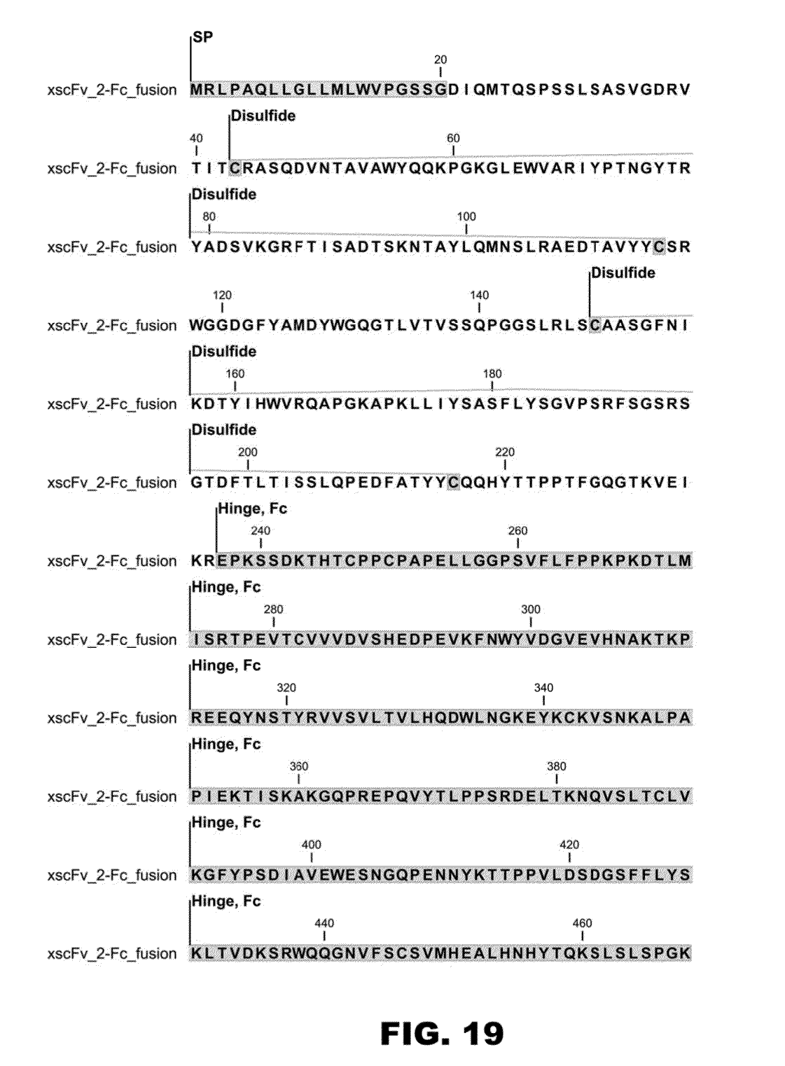

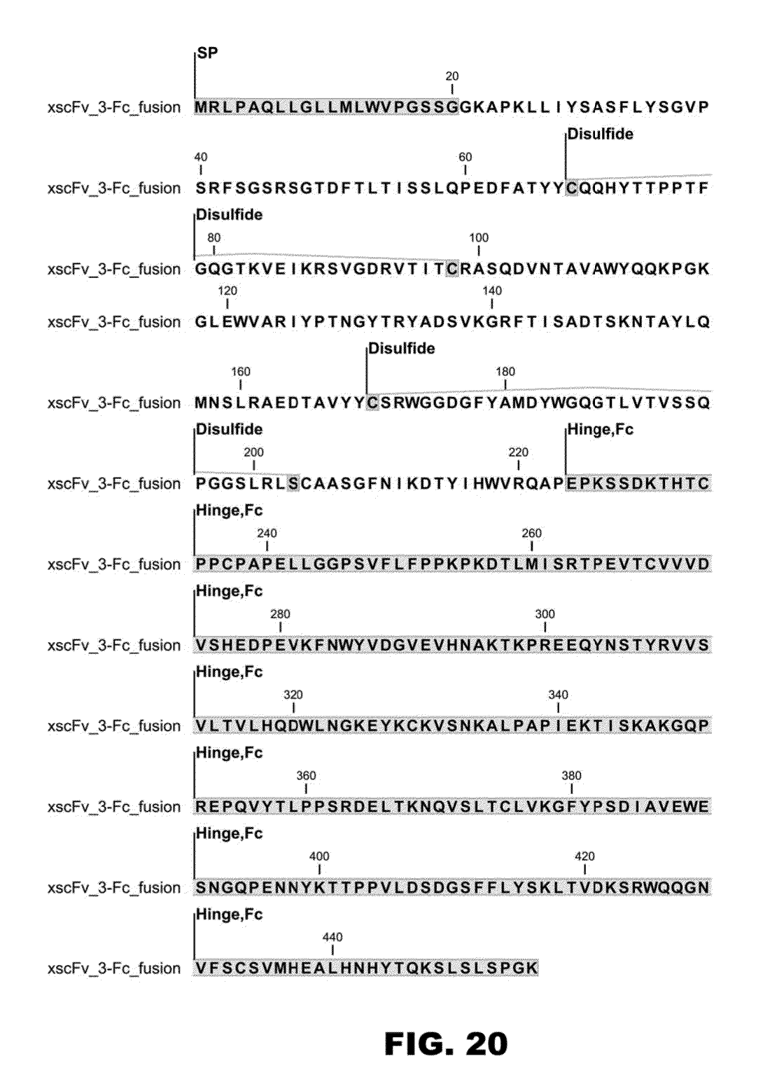

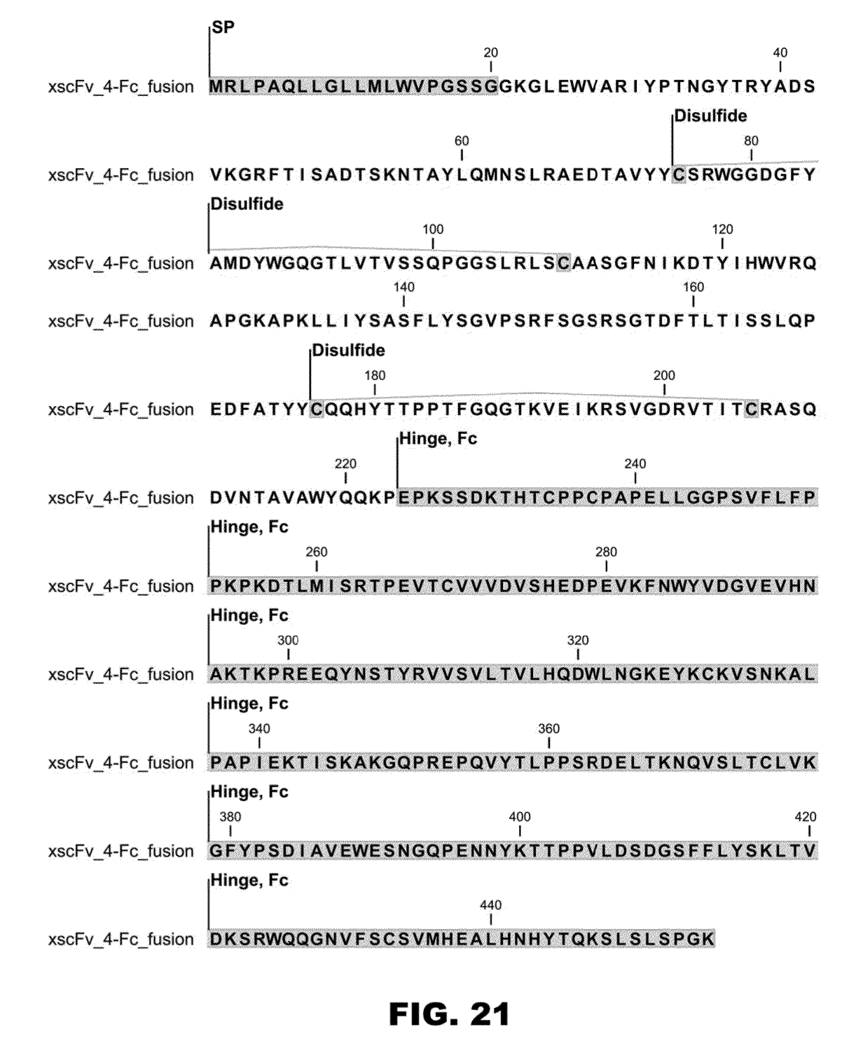

xscFv_1-Fc fusion protein (SEQ ID NO: 50) (see also FIG. 18)
MRLPAQLLGLLMLWVPGSSGEVQLVESGGGLVQPGGSLRLSCAASGFNIKDTYIHWVRQAPGKAPKLLIYSASFLYS
GVPSRFSGSRSGTDFTLTISSLQPEDFATYYCQQHYTTPPTFGQGTKVEIKRSVGDRVTITCRASQDVNTAVAWYQQ
KPGKGLEWVARIYPTNGYTRYADSVKGRFTISADTSKNTAYLQMNSLRAEDTAVYYCSRWGGDGFYAMDYWGQGTLV
TVSSEPKSSDKTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHEALHNHYTQKSLS
LSPGK
xscFv_2-Fc fusion protein (SEQ ID NO: 51) (see also FIG. 19)
MRLPAQLLGLLMLWVPGSSGDIQMTQSPSSLSASVGDRVTITCRASQDVNTAVAWYQQKPGKGLEWVARIYPTNGYT
RYADSVKGRFTISADTSKNTAYLQMNSLRAEDTAVYYCSRWGGDGFYAMDYWGQGTLVTVSSQPGGSLRLSCAASGF
NIKDTYIHWVRQAPGKAPKLLIYSASFLYSGVPSRFSGSRSGTDFTLTISSLQPEDFATYYCQQHYTTPPTFGQGTK
VEIKREPKSSDKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVMHEALHNHYTQKSL
SLSPGK
xscFv_3-Fc fusion protein (SEQ ID NO: 52) (see also FIG. 20)
MRLPAQLLGLLMLWVPGSSGGKAPKLLIYSASFLYSGVPSRFSGSRSGTDFTLTISSLQPEDFATYYCQQHYTTPPT
FGQGTKVEIKRSVGDRVTITCRASQDVNTAVAWYQQKPGKGLEWVARIYPTNGYTRYADSVKGRFTISADTSKNTAY
LQMNSLRAEDTAVYYCSRWGGDGFYAMDYWGQGTLVTVSSQPGGSLRLSCAASGFNIKDTYIHWVRQAPEPKSSDKT
HTCPPCPAPELLGGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQKSLSLSPGK
xscFv_4-Fc fusion protein (SEQ ID NO: 53) (see also FIG. 21)
MRLPAQLLGLLMLWVPGSSGGKGLEWVARIYPTNGYTRYADSVKGRFTISADTSKNTAYLQMNSLRAEDTAVYYCSR
WGGDGFYAMDYWGQGTLVTVSSQPGGSLRLSCAASGFNIKDTYIHWVRQAPGKAPKLLIYSASFLYSGVPSRFSGSR
SGTDFTLTISSLQPEDFATYYCQQHYTTPPTFGQGTKVEIKRSVGDRVTITCRASQDVNTAVAWYQQKPEPKSSDKT
HTCPPCPAPELLGGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQKSLSLSPGK

### Example 3

### Antibody Purification

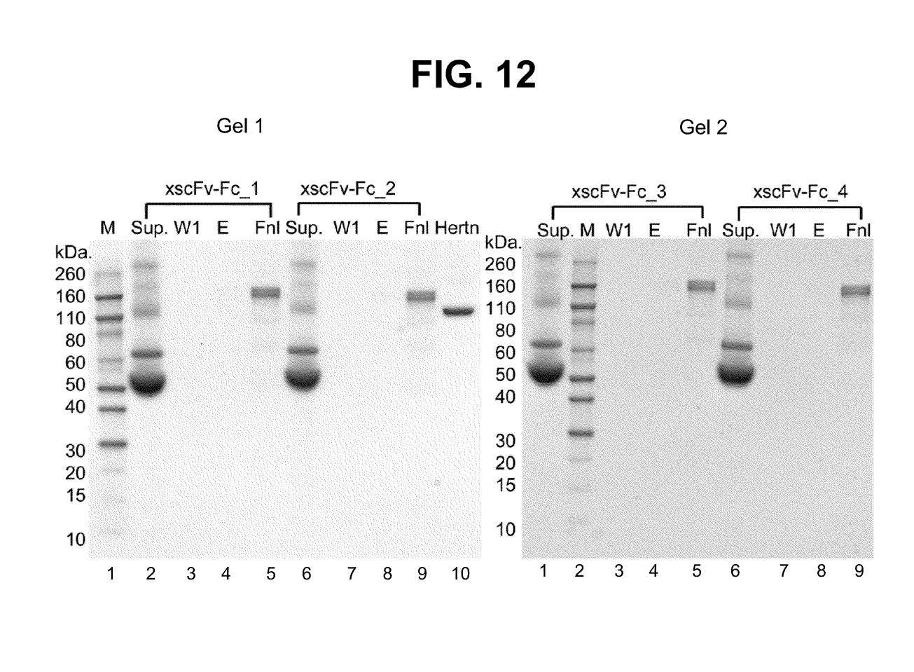

Cell culture (293T) supernatant containing the secreted antibody was incubated with pre-washed Protein A Sepharose beads (GE) at 4° C. overnight. The supernatant suspension was poured into a column the next day. The beads were allowed to settle and then washed once with 0.5 M NaCl/1×PBS buffer, followed by second wash using 1×PBS buffer. Antibody was eluted from protein A beads with low pH elution buffer (Pierce IgG elution buffer, pH 2.8) and washed with PBS. The purified xscFv-Fc antibodies were buffer exchanged using ultra filter device (Amicon) and concentrated to ˜1 mg/ml. An aliquot from each purification step was analyzed by SDS-PAGE. The final xscFv protein aliquots were stored in −80° C. freezer. Two-dimensional gels of resolved xscFvs are shown in FIG. 12.

### Example 4

### ELISA

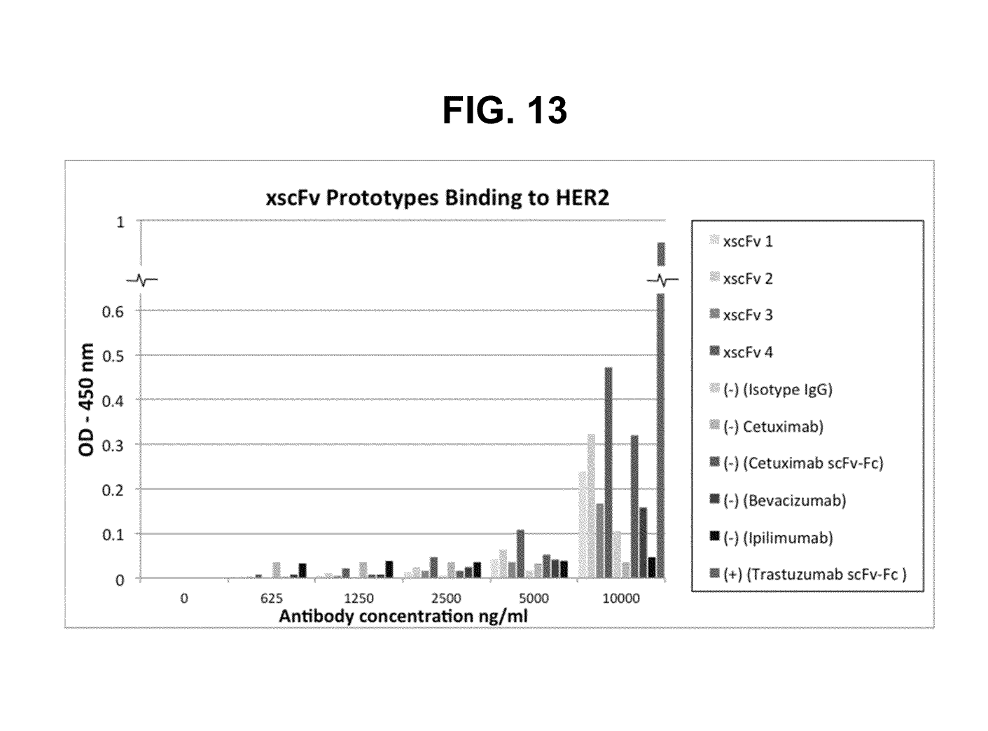

A 96 well maxi-absorb plate was coated with HER2 antigen (2.5 μs) and incubated at 4° C. overnight. Unbound proteins were washed away with PBST (0.05% Tween 20) the next day. The plate was blocked with 2% BSA/PBS for 2 hours at room temperature, followed by six PBST washes. The xscFv-Fc antibodies and the appropriate positive and negative control antibodies were diluted and incubated for 1 hour at room temperature. The plate was washed again in PBST and incubated with secondary antibody (HRP-Human Fc-Southern Biotech) in 1:10,000 dilution for 30 minutes at room temperature. 100 μl of substrate solution was added until color developed after the final washes in PBST. The reaction was stopped using 100 μl/well Stop Solution (KPL) and after 5 minutes of equilibration at room temperature, the plate was read at OD 450 nm wavelength. XscFv binding patterns are demonstrated in FIG. 13.

### Example 5

### Design of xscFv Libraries

Standard comparative modeling techniques using Discovery Studio (Biovia, San Diego, Calif.) are used to construct models of monoclonal V domains. Sequences of unknown structure are compared to multiple sequences with known structures. The sequences which align best are used as templates for model building. The atomic coordinates of identical residues and backbone atoms are kept, and additional residues are inserted or deleted as indicated by the sequence alignment. In cases where loops must be constructed, loop libraries of sizes specific to the build are interrogated for idealized structures. The resulting models are energy minimized to optimize residue positions, bond lengths, angles, bonded and non-bonded interactions, and electrostatics.

References describing 2-dimensional IMGT Collier de Perles numbering and depictions include:
Ruiz, M. and Lefranc, M.-P., “IMGT gene identification and Colliers de Perles of human immunoglobulins with known 3D structures”, Immunogenetics, 53, 857-883 (2002). PMID: 11862387;
Kaas, Q. and Lefranc, M.-P., “IMGT Colliers de Perles: Standardized Sequence-Structure Representations of the IgSF and MhcSF Superfamilly Domains”, Current Bioinformatics, 2, 21-30 (2007);
Kaas, Q., Ehrenmann, F. and Lefranc, M.-P., “IG, TR and IgSF, MHC and MhcSF: what do we learn from the IMGT Colliers de Perles?”, Brief. Funct. Genomic Proteomic, 6, 253-264 (2007). PMID: 18208865; and,
Ehrenmann, F., Giudicelli., V, Duroux, P., Lefranc, M.-P., “IMGT/Collier de Perles: IMGT standardized representation of domains (IG, TR, and IgSF variable and constant domains, MH and MhSF groove domains)”, Cold Spring Harbor Protoc., 6, 726-736 (2011). PMID: 21632776.
   Each of the above-cited references are hereby incorporated by reference.
See also, “Make your own IMGT/Collier de Perles” web-based software for generating IMGT numbered VH and VL 2-dimensional depictions at www.imgt.org/3Dstructure-DB/cgi/Collier-de-Perles.cgi

1. A single chain antibody polypeptide comprising a heavy chain variable domain and a light chain variable domain, wherein the antibody polypeptide comprises at least one interdomain crossover, at least one intradomain crossover, or at least one intradomain crossover and at least one interdomain crossover, wherein each crossover is an engineered peptide bond producing an intercalated structure (arrangement or organization) selected from the group consisting of:
a) at least one portion of the heavy chain variable domain intercalated (inserted) into the light chain variable domain;
b) at least one portion of the light chain variable domain intercalated (inserted) into the heavy chain variable domain;
c) at least one portion of the light chain variable domain intercalated (inserted) into a different portion of the light chain variable domain; and,
d) at least one portion of the heavy chain variable domain intercalated (inserted) into a different portion of the heavy chain variable domain.

2. The single chain antibody polypeptide of claim 1, comprising at least one intradomain crossover and at least one interdomain crossover.

3. The single chain antibody polypeptide of claim 1, comprising at least two intradomain crossovers.

4. The single chain antibody polypeptide of claim 1, wherein each crossover is introduced between variable domain regions which are within approximately 9-12 Å of each other prior to intercalation (insertion).

5. The single chain antibody polypeptide of claim 1, wherein the intercalated (inserted) portion comprises at least one variable domain immunoglobulin beta strand.

6. The single chain antibody polypeptide of claim 5, wherein said at least one variable domain immunoglobulin beta strand is intercalated (inserted) within a different region (portion) of the same variable domain.

7. The single chain antibody polypeptide of claim 1, wherein the polypeptide comprises immunoglobulin beta strands (1)-(17) arranged sequentially from the N to the C terminus in the polypeptide, wherein the immunoglobulin beta strands (1)-(17) are selected from the group consisting of:
(i) 1) A_{H}; 2) B_{H}; 3) C_{H}; 4) C′_{L}; 5) C″_{L}; 6) D_{L}; 7) E_{L}; 8) F_{L}; 9) G_{L}; 10) B_{L}; 11) C_{L}; 12) C′_{H}; 13) C″_{H}; 14) D_{H}; 15) E_{H}; 16) F_{H}; and 17) G_{H}; and
(ii) 1) A_{L}; 2) B_{L}; 3) C_{L}; 4) C′_{H}; 5) C″_{H}; 6) D_{H}; 7) E_{H}; 8) F_{H}; 9) G_{H}; 10) B_{H}; 11) C_{H}; 12) C′_{L}; 13) C″_{L}; 14) D_{L}; 15) E_{L}; 16) F_{L}; and 17) G_{L}.

8. The single chain antibody polypeptide of claim 7, wherein immunoglobulin beta strands (1)-(17) are selected from the group consisting of:
a) A_{L} comprises amino acid residues DIQMTQSPSSLSASV (SEQ ID NO:1);
b) B_{L} comprises amino acid residues GDRVTITCRASQDV (SEQ ID NO:2);
c) C_{L} comprises amino acid residues NTAVAWYQQKP (SEQ ID NO:3);
d) C′_{L} comprises amino acid residues GKAPKLLIYSA (SEQ ID NO:4);
e) C″_{L} comprises amino acid residues SFLYSGVPS (SEQ ID NO:5);
f) D_{L} comprises amino acid residues RFSGSRSG (SEQ ID NO:6);
g) E_{L} comprises amino acid residues TDFTLTISSLQP (SEQ ID NO:7);
h) F_{L} comprises amino acid residues EDFATYYCQQHYT (SEQ ID NO:8);
i) G_{L} comprises amino acid residues TPPTFGQGTKVEIK (SEQ ID NO:9);
j) G_{L} comprises amino acid residues TPPTFGQGTKVEIKR (SEQ ID NO:10);
k) A_{H} comprises amino acid residues EVQLVESGGGLVQP (SEQ ID NO:11);
l) B_{H} comprises amino acid residues GGSLRLSCAASGFNI (SEQ ID NO:12);
m) C_{H} comprises amino acid residues KDTYIHWVRQAP (SEQ ID NO:13);
n) C′_{H} comprises amino acid residues GKGLEWVARIYPT (SEQ ID NO:14);
o) C″_{H} comprises amino acid residues NGYTRYADSVKG (SEQ ID NO:15);
p) D_{H} comprises amino acid residues RFTISADTSK (SEQ ID NO:16);
q) E_{H} comprises amino acid residues NTAYLQMNSLRA (SEQ ID NO:17);
r) F_{H} comprises amino acid residues EDTAVYYCSRWGGDG (SEQ ID NO:18);
s) G_{H} comprises amino acid residues FYAMDYWGQGTLVTVSS (SEQ ID NO:19); and,
t) G_{H} comprises amino acid residues FYAMDYWGQGTLVTVSSQP (SEQ ID NO:20).

9. The single chain antibody polypeptide of claim 1, wherein the polypeptide comprises immunoglobulin beta strands (1)-(16) arranged sequentially from the N to the C terminus in the polypeptide, wherein the immunoglobulin beta strands (1)-(16) are selected from the group consisting of
(i) 1) C′_{L}; 2) C″_{L}; 3) D_{L}; 4) E_{L}; 5) F_{L}; 6) G_{L}; 7) B_{L}; 8) C_{L}; 9) C′_{H}; 10) C″_{H}; 11) D_{H}; 12) E_{H}; 13) F_{H}; 14) G_{H}; 15) B_{H}; and 16) C_{H}; and
(ii) 1) C′_{H}; 2) C″_{H}; 3) D_{H}; 4) E_{H}; 5) F_{H}; 6) G_{H}; 7) B_{H}; 8) C_{H}; 9) C′_{L}; 10) C″_{L}; 11) D_{L}; 12) E_{L}; 13) F_{L}; 14) G_{L}; 15) B_{L}; and 16) C_{L}.

10. The single chain antibody polypeptide of claim 9, wherein immunoglobulin beta strands (1)-(16) are selected from the group consisting of:
a) A_{L} comprises amino acid residues DIQMTQSPSSLSASV (SEQ ID NO:1);
b) B_{L} comprises amino acid residues GDRVTITCRASQDV (SEQ ID NO:2);
c) C_{L} comprises amino acid residues NTAVAWYQQKP (SEQ ID NO:3);
d) C′_{L} comprises amino acid residues GKAPKLLIYSA (SEQ ID NO:4);
e) C″_{L} comprises amino acid residues SFLYSGVPS (SEQ ID NO:5);
f) D_{L} comprises amino acid residues RFSGSRSG (SEQ ID NO:6);
g) E_{L} comprises amino acid residues TDFTLTISSLQP (SEQ ID NO:7);
h) F_{L} comprises amino acid residues EDFATYYCQQHYT (SEQ ID NO:8);
i) G_{L} comprises amino acid residues TPPTFGQGTKVEIK (SEQ ID NO:9);
j) G_{L} comprises amino acid residues TPPTFGQGTKVEIKR (SEQ ID NO:10);
k) A_{H} comprises amino acid residues EVQLVESGGGLVQP (SEQ ID NO:11);
l) B_{H} comprises amino acid residues GGSLRLSCAASGFNI (SEQ ID NO:12);
m) C_{H} comprises amino acid residues KDTYIHWVRQAP (SEQ ID NO:13);
n) C′_{H} comprises amino acid residues GKGLEWVARIYPT (SEQ ID NO:14);
o) C″_{H} comprises amino acid residues NGYTRYADSVKG (SEQ ID NO:15);
p) D_{H} comprises amino acid residues RFTISADTSK (SEQ ID NO:16);
q) E_{H} comprises amino acid residues NTAYLQMNSLRA (SEQ ID NO:17);
r) F_{H} comprises amino acid residues EDTAVYYCSRWGGDG (SEQ ID NO:18);
s) G_{H} comprises amino acid residues FYAMDYWGQGTLVTVSS (SEQ ID NO:19); and,
t) G_{H} comprises amino acid residues FYAMDYWGQGTLVTVSSQP (SEQ ID NO:20).

11. An antibody framework comprising the single chain antibody polypeptide of claim 1, wherein the antibody framework is selected from the group consisting of: a Fab fragment comprising crossovers, a F(ab′)₂ fragment comprising crossovers, an Fv fragment comprising crossovers, a diabody comprising crossovers, a minibody comprising crossovers, a bispecific antibody comprising crossovers, a bispecific single-chain Fvs (bsscFvs) comprising crossovers, and a chimeric antigen receptor comprising crossovers.

12. The single chain antibody polypeptide of claim 1, wherein the antibody polypeptide is an xscFv.

13. The single chain antibody polypeptide of claim 1, wherein the polypeptide further comprises a linker of 1, 2, 3, 4, 5, 6, 7, or 8 amino acid residues.

14. A nucleic acid encoding the single chain antibody polypeptide of claim 1.

15. A vector comprising the nucleic acid of claim 14.

16. A host cell comprising the nucleic acid of claim 14.

17. A host cell expressing the single chain antibody polypeptide of claim 1.

18. A method of making a single chain antibody polypeptide comprising culturing the host cell of claim 17 under conditions supporting antibody polypeptide expression.

19. An in vitro method of targeting an antigen comprising contacting an antigen in vitro with the single chain antibody, polypeptide of claim 1 under conditions wherein said single chain antibody polypeptide binds said antigen.

20. An in vivo method of targeting an antigen comprising contacting an antigen in vivo with an antibody polypeptide of claim 1 under conditions wherein said single chain antibody polypeptide binds said antigen.

21. A pharmaceutical composition or medicament comprising the single chain antibody polypeptide of claim 1.

22. A library of single chain antibody polypeptides, wherein said single chain antibody polypeptides comprise a heavy chain variable domain and a light chain variable domain, wherein the antibody polypeptides comprise at least one interdomain crossover, at least one intradomain crossover, or at least one intradomain crossover and at least one interdomain crossover, wherein each crossover is selected from the group consisting of:
a) at least one portion of a heavy chain variable domain intercalated (inserted) into a light chain variable domain;
b) at least one portion of a light chain variable domain intercalated (inserted) into a heavy chain variable domain;
c) at least one portion of a light chain variable domain intercalated (inserted) into a different portion of the light chain variable domain; and,
d) at least one portion of a heavy chain variable domain intercalated (inserted) into a different portion of the heavy chain variable domain.
